(19) United States
(12) Patent Application Publication
(10) Pub. No.: US 2021/0352950 A1
(43) Pub. Date: Nov. 18, 2021

(54) FASTING MIMICKING DIET AND VITAMIN C FOR THE TREATMENT OF CANCER

(71) Applicants: Valter LONGO, Milano (MI) (IT); Maira DI TANO, Milano (MI) (IT)

(72) Inventors: Valter LONGO, Milano (MI) (IT); Maira DI TANO, Milano (MI) (IT)

(21) Appl. No.: 17/387,165

(22) Filed: Jul. 28, 2021

Related U.S. Application Data
(63) Continuation of application No. PCT/EP2020/052010, filed on Jan. 28, 2020.

(30) Foreign Application Priority Data
Jan. 28, 2019 (EP) 19153957.6

(51) Int. Cl. A23L 33/00; A23L 33/15; A61K 31/375; A61K 45/06; A61P 35/00

(52) U.S. Cl. CPC A23L 33/30; A23L 33/15; A61P 35/00; A61K 45/06; A61K 31/375

(57) ABSTRACT

The present invention relates to the combination of a Fasting Mimicking Diet (FMD) and vitamin C (ascorbic acid) for use in the treatment of cancer. The combination is particularly useful in the treatment of KRAS mutant solid cancers.

### CROSS-REFERENCE TO RELATED APPLICATIONS

This application is a bypass continuation of PCT/EP2020/052010, filed Jan. 28, 2020, which claims the benefit of European Patent Application No. 19153957.6, filed Jan. 28, 2019, the contents of each of which are incorporated herein by reference.

### FIELD OF THE INVENTION

The present invention relates to the combination of a Fasting Mimicking Diet (FMD) and vitamin C (ascorbic acid) for use in the treatment of cancer. The combination is particularly useful in the treatment of KRAS mutant cancers.

### BACKGROUND TO THE INVENTION

RAS genes represent the most frequently mutated oncogene family in human cancer and so far, all attempts to selectively drug RAS signalling have failed in the clinic (Cox et al., 2014; Stephen et al. 2014; according to COSMIC (catalogue of somatic mutation in cancer)).

RAS mutant tumors are associated with a poor prognosis, due to their unresponsiveness to the majority of standard and targeted therapies, for this reason a major effort has been devoted to identifying specific RAS inhibitors. However, all the past studies have failed in achieving this, creating the idea that the target is “undraggable” (Lievre et al., 2006; Cox et al., 2014; Verissimo et al., 2016). KRAS mutations are found in the three most lethal cancers: colorectal cancer (in 30-50% of cases), lung cancer (in almost 30% of cases) and pancreatic cancer (in more than 90% of cases) (Bardelli and Siena, 2010; Cox et al., 2014). A recent work has shown that high doses of ascorbic acid (vitamin C) selectively kills KRAS mutant colorectal cancer (CRC) in different mouse models (Yun et al., 2015). A large body of evidence showed that vitamin C exerts its anti-tumoral effect by acting as pro-oxidant agent and causing the formation of hydrogen peroxide. Hydrogen peroxide accumulation generates hydroxyl radicals by Fenton reaction chemistry, finally causing oxidative damage and cell death (Chen et al., 2008; Du et al., 2012; Ma et al., 2014; Yun et al., 2015; Aguilera et al., 2016; Shoenfeld et al., 2017). However, vitamin C as single intervention shows limited efficacy in pre-clinical and clinical studies. For this reason, it is of fundamental importance the identification of a non-toxic intervention, which could potentiate vitamin C anti-cancer effect.

Fasting and fasting-mimicking diet (FMD) was shown to reduce tumor progression and sensitize different types of cancer to chemotherapy while protecting normal cells from chemo-associated toxic effects (Lee et al, 2012). These phenomena are known as “Differential Stress Sensitization” (DSS) and “Differential Stress Resistance” (DSR), respectively (Raffaghello et al., 2008; Safdie et al., 2009; Lee et al., 2010; Lee et al, 2012: Longo and Mattson, 2014). The differential protection of normal and cancer cells in response to fasting has been proposed to be mediated, at least in part, by regulating the insulin-growth factor (IGF-1) pathway (Lee et al., 2010). By decreasing IGF-1 level, fasting allows normal cells to enter in a protection mode, reducing mitotic stimuli and inducing the expression of repair genes (Longo and Mattson, 2014; Raffaghello et al., 2008). Cancer cells, instead, harbouring oncogenic mutations, are unresponsive to growth signals, and thus fail to be protected (Lee et al., 2012; Lee et al., 2010). However, fasting remains a challenging option for cancer patients, for this reason the inventors developed a more feasible and safer diet whose specific formulation mimics fasting effects. Fasting mimicking diet (FMD) is low in calories, proteins and sugars but high in unsaturated fats and, as fasting, it is able to reduce the level of cancer risk factors such as glucose and IGF-1, which are the major players involved in DSS and DSR (Brandhorst et al., 2015; Wei et al., 2017).

A growing body of studies showed that high dose vitamin C is selectively toxic to cancer cells, without affecting normal tissues; thus, representing an important opportunity for a safe and well tolerated anti-cancer treatment (Schoenfeld et al., 2017). Moreover, it has been found that KRAS mutant cancers show a higher susceptibility to pharmacological doses of vitamin C compared to KRAS wild type tumors; however, its therapeutic potential result to be limited (Yun et al., 2015; Padayatty et al., 2010; Ma et al., 2014).

There is still the need for a treatment of cancer, in particular of KRAS-driven cancers or solid cancers, especially of KRAS mutant solid cancers, which is effective and well-tolerated.

### SUMMARY OF THE INVENTION

In the present invention, the authors have identified the combination of a specific caloric intake regime with vitamin C (ascorbic acid) for use in the treatment of cancer. Said a specific caloric intake regime is based on a reduced daily caloric intake compared to a regular daily caloric intake, in particular it involves a specific daily caloric intake and a specific macronutrient intake as defined below.

The combination of the present invention is particularly advantageous in that it enhances the anti-cancer effect of vitamin C when administered alone. In particular, the specific caloric intake regime of the invention sensitises cancer cells to vitamin C toxicity. Specifically, the combination of the invention is able to delay tumor progression. The combination of the invention also potentiates the efficacy of further therapeutic interventions, such as chemotherapy. The combination of the invention is particularly advantageous also because it is safe and well tolerated. Surprisingly and advantageously, the combination of the invention is effective on KRAS mutant solid cancer.

In the present invention it is shown that vitamin C alone has a relatively mild toxic effect against CRC, in part caused by the up-regulation of the stress-inducible protein heme-oxygenase-1 (HO-1). FMD can cause a major enhancement in vitamin C toxicity in KRAS mutant CRC both in vitro and in vivo. The mechanism underlying this effect involves, at least in part, the FMD-dependent downregulation of HO-1. It is presently shown that FMD, by reverting the HO-1 induction mediated by vitamin C, is able to reduce ferritin level, leading to an increase in free ferrous ions (Fe2+). The increase of Fe2+, together with a dramatic enhancement of ROS generation, leads to DNA damage and cell death, most likely through Fenton reaction.

Collectively, the present invention shows that FMD represents a safe therapeutic intervention, able to potentiate vitamin C anti-cancer effect. In addition, evidence indicating that FMD and vitamin C combination therapy enhances oxaliplatin efficacy in KRAS-driven CRC mouse models is provided, thus representing a valuable therapeutic option.

It is therefore an object of the invention a reduced caloric intake or a fasting mimicking diet and vitamin C for use in the treatment of cancer.

Preferably, said reduced caloric intake or fasting mimicking diet lasts for a period of 24 to 190 hours. Preferably, said reduced caloric intake or fasting mimicking diet it lasts for a period of 24 to 120 hours. Preferably, said reduced caloric intake or fasting mimicking diet lasts for approximately 5 days.

Preferably, said reduced caloric intake or fasting mimicking diet is a regular caloric intake reduced by 10% to 100%. Preferably, said reduced caloric intake or fasting mimicking diet is a regular caloric intake reduced by 45% to 95%. Preferably, said reduced caloric intake or fasting mimicking diet is a regular caloric intake reduced by approximately 50% to 70%.

Preferably, said reduced caloric intake or fasting mimicking diet is 0 to 90% of the regular caloric intake. Preferably, said reduced caloric intake or fasting mimicking diet is 5% to 55% of the regular caloric intake. Preferably, said reduced caloric intake or fasting mimicking diet is approximately 50% to 30% of a regular caloric intake.

Preferably, said reduced caloric intake or fasting mimicking diet comprises a first period of 0 to 24 hours wherein caloric intake is a regular caloric intake reduced by 40-60%, followed by a second period of 24 to 144 hours wherein caloric intake is a regular caloric intake reduced by 60-95%.

Preferably, the caloric intake in the first period is a regular caloric intake reduced by approximately 50%. Preferably, the caloric intake in the second period is a regular caloric intake reduced by approximately from 65 to 95% (i.e. the caloric intake in the second period is approximately from 5% to 35% of the regular caloric intake). Preferably, the caloric intake in the second period is a regular caloric intake reduced by approximately from 70 to 90% (i.e. the caloric intake in the second period is approximately from 10% to 30% of the regular caloric intake). Preferably, the caloric intake in the second period is a regular caloric intake reduced by approximately 70% (i.e. the caloric intake in the second period is approximately 30% of the regular caloric intake).

Preferably, said first period lasts approximately 24 hours. Preferably, said second period lasts approximately from 48 to 96 hours. Also preferably, said second period lasts approximately from 24 to 48 hours, or approximately from 24 to 72 hours, or approximately from 24 to 96 hours. Preferably, said second period lasts 24, 48, 72 or 96 hours.

Preferably, said reduced caloric intake or fasting mimicking diet comprises a reduced protein intake and/or a reduced simple carbohydrate intake and/or an increased complex carbohydrate intake and/or an increased unsaturated fat intake. More preferably, said reduced caloric intake or fasting mimicking diet comprises a reduced protein intake, a reduced simple carbohydrate intake, an increased complex carbohydrate intake and an increased unsaturated fat intake.

Preferably, said reduced protein intake is from 5 to 15% of total caloric intake. More preferably said reduced protein intake is approximately from 9 to 11% of total caloric intake. Preferably, said increased complex carbohydrate intake is from 40 to 50% of total caloric intake. More preferably, said increased complex carbohydrate intake is approximately from 43 to 47% of total caloric intake. Preferably, said increased unsaturated fat intake is from 40 to 50% of total caloric intake. More preferably, said increased unsaturated fat intake is approximately from 44 to 46% of total caloric intake.

Preferably, vitamin C is administered parenterally. More preferably, vitamin C is administered intravenously.

Preferably, vitamin C is administered in an amount of approximately from 50 to 100 g. Preferably, said vitamin C is administered three times per week.

Preferably, said reduced caloric intake or fasting mimicking diet and vitamin C are combined with a further therapeutic intervention.

Preferably, said further therapeutic intervention is selected from the group consisting of: surgery, radiotherapy and a further therapeutic agent.

Preferably, said further therapeutic agent is a chemotherapeutic agent. Preferably, said chemotherapeutic agent is selected from the group consisting of: a DNA synthesis inhibitor, a monoclonal antibody and a Heme Oxygenase-1 (HO-1) inhibitor. Preferably, said monoclonal antibody is a monoclonal antibody directed against EGFR. Preferably, said chemotherapeutic agent is selected from the group consisting of: oxaliplatin, zinc protoporphyrin, 5-fluorouracil (5-FU), folinic acid, irinotecan, capecitabine, cetuximab, panitumumab, bevacizumab, FOLFOX, FOLFOXIRI, XELIRI, and XELOX.

In a preferred embodiment, the reduced caloric intake or the fasting mimicking diet and vitamin C as defined above are for use in combination with oxaliplatin and/or zinc protoporphyrin.

Preferably, said cancer is a solid cancer. Also preferably, said cancer is a RAS mutant cancer. Still preferably said cancer is a KRAS mutant cancer.

Preferably, said cancer is resistant to radiotherapy or chemotherapy. Preferably, said cancer is resistant to: a DNA synthesis inhibitor, a monoclonal antibody and/or a Heme Oxygenase-1 (HO-1) inhibitor. Preferably, said cancer is resistant to a monoclonal antibody directed against EGFR. Preferably, said cancer is resistant to: oxaliplatin, zinc protoporphyrin, 5-fluorouracil (5-FU), folinic acid, irinotecan, capecitabine, cetuximab, panitumumab, bevacizumab, FOLFOX, FOLFOXIRI, XELIRI, and/or XELOX.

Preferably, said cancer is selected from the group consisting of: colorectal cancer, lung cancer, pancreatic cancer, colon cancer, rectal cancer, mucinous adenocarcinoma.

Preferably, said cancer is a metastatic cancer.

In a preferred embodiment, the reduced caloric intake or the fasting mimicking diet and vitamin C as defined above are for use in the treatment of a solid KRAS mutant cancer.

Preferably, said cancer is a KRAS mutant solid cancer. Preferably, said KRAS mutant solid cancer is selected from the group consisting of: a KRAS mutant colorectal cancer, a KRAS mutant lung cancer, a KRAS mutant pancreatic cancer, a KRAS mutant colon cancer, a KRAS mutant rectal cancer and a KRAS mutant mucinous adenocarcinoma.

Preferably, said reduced caloric intake or fasting mimicking diet and vitamin C increase cellular oxidative stress and/or increase cellular iron content.

In the present invention, a specific caloric and macronutrient intake may be achieved, for example, by means of fasting or of a fasting mimicking diet (FMD).

Fasting involves 2-4 days of starvation, with free consumption to water.

“Fasting mimicking diet” (FMD) refers to previously described formulations to mimic the effects of fasting. Complete fasting results to be challenging for cancer patients, especially when undergoing chemotherapy, so the inventors have developed a FMD that enables a patient to eat “food” while achieving the same effects of fasting on normal and cancer cells.

The fasting or FMD is started one day before the therapy and continues for the following 2-4 days while the therapy is most active.

In particular, FMD comprises one or more FMD cycles, each cycle consisting of 2-5 days (preferably of 2-4 days) of low-calorie intake as follows:

Day 1:

Mouse=50% of regular calorie intake

Human=50% of regular calorie intake

Days 2-5:

Mouse=10% of regular calorie intake (i.e. regular calorie intake reduced by 90%)

Human=30% of regular calorie intake (i.e. regular calorie intake reduced by 70%).

FMD is achieved with a low protein and low sugar and high fat plant-based formulation followed by a standard/regular ad libitum diet until the complete recovery of bodyweight.

The reduction is compared to a regular caloric intake per day. Regular caloric intake per day is between 1200 Kcal and 3000 Kcal. Preferably regular caloric intake per day (the range is based on age, sex and fisical activity) is:

Age 4-8 years: 1200-2000 Kcal

Age 9-13 years: 1800-2600 Kcal

Age 19-30 years: 1800-3000 Kcal

Age 31-50 years: 1800-2600 Kcal

+51 years: 1600-2600 Kcal.

Usually, humans undergoing one or more FMD cycles do not lose more than 10% of bodyweight. FMD cycles are feasible and safe thus individuals well tolerate the diet.

Reasons for stopping the cycles are not related to heathy status but usually to non-compliance to the dietary protocol or for work scheduling issues. However, there are clinical condition which can make the individual not eligible to FMD, such as being underweight.

In a preferred embodiment, FMD is a 5-days regimen and vitamin C is administred intravenously (50-100 g) three times per week as previously described, starting from the second day of each FMD cycle.

In patients, intravenous doses of 50 to 100 g of Vitamin C three times per week can be safely administered to reach potentially active blood concentration of the compound (Padayatty et al., 2010; Monti et al., 2012; Ma et al., 2014; Schoenfeld et a., 2017).

Preferably the FMD or reduced caloric intake starts at least 24 hours before vitamin C is administered. Preferably the FMD or reduced caloric intake starts at least 48 hours before vitamin C is administered. Preferably the FMD or reduced caloric intake starts at least 96 hours before vitamin C is administered.

Preferably the FMD or reduced caloric intake lasts at least 24 hours after vitamin C is administered. Preferably the FMD or reduced caloric intake lasts at least 72 hours after vitamin C is administered. Preferably the FMD or reduced caloric lasts at least 48, 72, 96, 120 hours after vitamin C is administered.

Preferably the FMD or reduced caloric intake starts one day before vitamin C is administered and continues for the following 2-4 days while vitamin C is also administered. Preferably the FMD or reduced caloric intake consists of 5 days of low-calorie intake (50% of regular calorie intake on day 1, and 30% on days 2-5).

In a preferred embodiment, the FMD or reduced caloric intake and vitamin C for use according to the invention increase cellular oxidative stress. In the present invention, an increase in cellular oxidative stress refers to an increase in Reactive Oxigen Species production, as measured by the oxidation of a fluorigenic probe. For instance, CellRox reagent may be used as fluorigenic probe. CellROX fluorogenic probe is designed to measure reactive-oxygen species (ROS) in live cells. The probe is cell permeable and in reduced state it is no or weakly fluorescent, whereas upon oxidation it shows a fluorogenic signal. CellROX probe exhibits a fluorescence excitation at 640 nm and fluorescent emission at 665 nm (deep red).

In a preferred embodiment, the FMD or reduced caloric intake and vitamin C for use according to the invention increase cellular iron content. In the present invention, an increase in cellular iron content refers to
the level of free iron pool measured by a colorimentric method: Ferrous ions (Fe2+), but not ferric ions (Fe3+) specifically reacts with ferene-S (an iron chromogen) producing a stable colored complex, whose absorbance is measured at 593 nm;
the expression level of proteins involved in iron metabolism, in particular ferritin expression level.

The present invention will be illustrated by means of non-limiting examples and figures as follows.

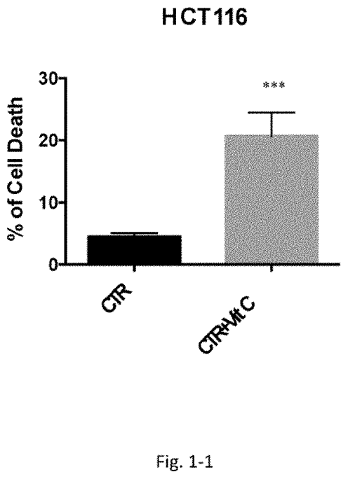

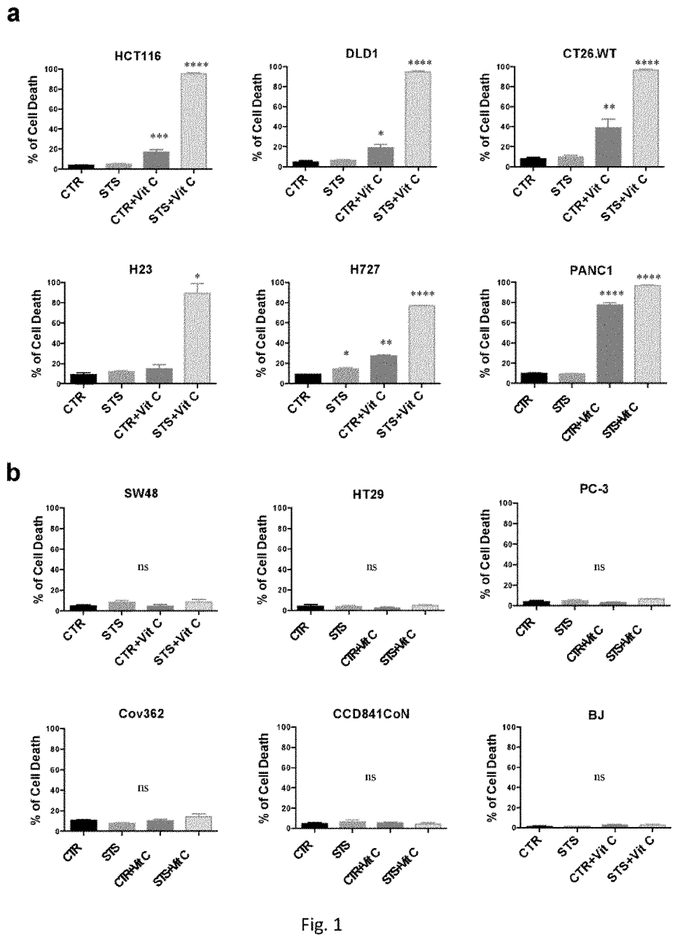

FIG. 1-1. Pharmacological vitamin C induces cell death in HCT116. HCT116 cells were grown in control (CTR) condition medium and were treated with vitamin C (Vit C; 350 μM) or vehicle for 24 hours. Percentage of cell death was assessed by Muse Cell Viability analyser (n≥5 biological replicates). Data are represented as mean±SEM (two-tailed unpaired t-test; ***p value≤0.001).

FIG. 1. STS selectively sensitizes KRAS mutant cancer cells to vitamin C toxicity

KRAS mutant (A), KRAS wild type cancer cells and normal cells (B) were grown in CTR or STS conditions for a total of 48 hours. At 24 h, cells were treated with vitamin C (350 μM) or vehicle for further 24 hours. At 48 hours, viability was assessed by Muse Cell viability analyser. Data are represented as mean±SEM (two-tailed unpaired t test, * p value≤0.05, **p value≤0.01, ***p value≤0.001 ****p value≤0.0001, ns, not significant).

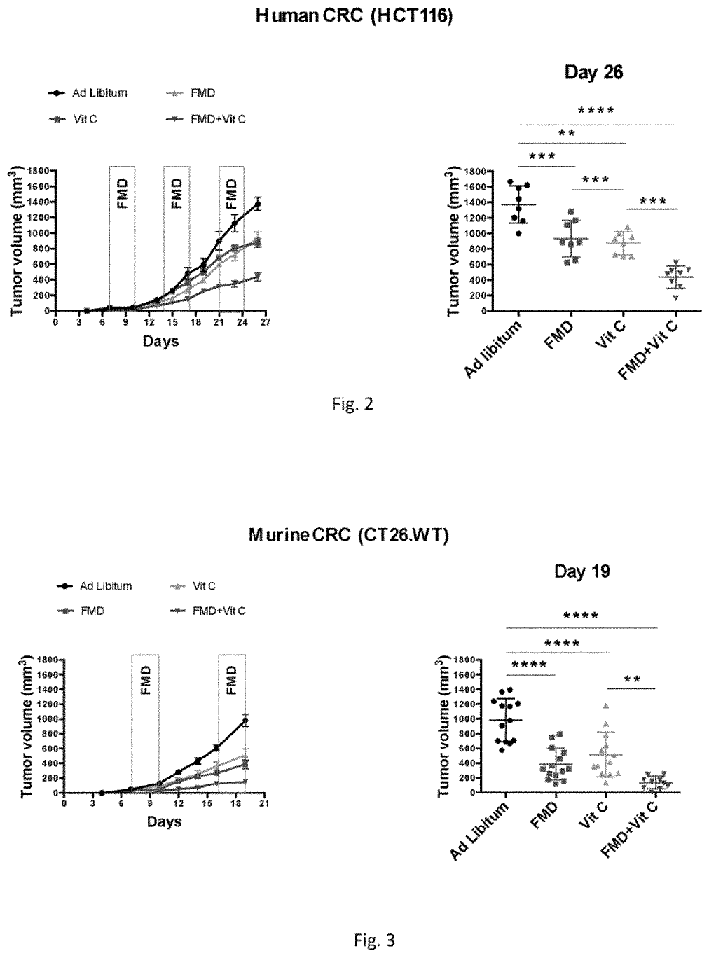

FIG. 2. A Fasting-Mimicking Diet (FMD) enhances vitamin C toxicity to reduce tumor progression in HCT116 xenograft. 8-weeks old female NOD scid gamma (NSG) mice were subcutaneously injected with HCT116 cells and subjected to 3 cycles of FMD alone or in combination with high dose vitamin C (Vit C; 4 g/kg twice a day, i.p.). Tumor volumes at multiple time points (left panel) and before mice were euthanized (right panel) are reported (n=8 per group). Data are represented as mean±SEM. One-way Anova (Tukey's post-analysis test) was performed (**p value≤0.01, ****p value≤0.0001).

FIG. 3. A Fasting-Mimicking Diet (FMD) enhances vitamin C toxicity in reducing tumor progression in CT26.WT allograft. 8-week old female Balb/c mice were subcutaneously injected with CT26.WT cells and subjected to 2 cycles of FMD alone or in combination with high dose vitamin C (Vit C; 4 g/kg twice a day, i.p.). Tumor volumes at multiple time points (left panel) and before mice were euthanized (right panel) are reported (n=10-14 per group). Data are represented as mean±SEM. One-way Anova (Tukey's post-analysis test) was performed (*p value≤0.05, **p value≤0.01, ****p value≤0.0001).

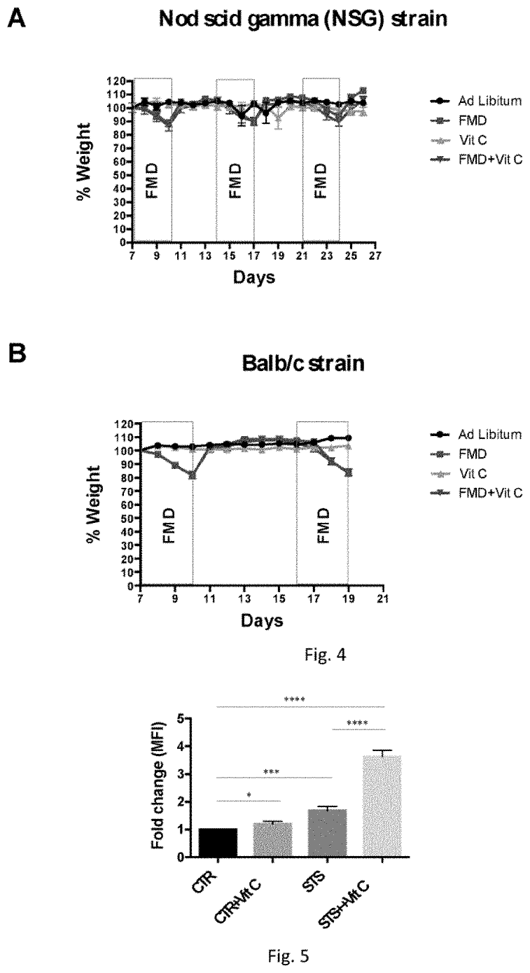

FIG. 4. FMD and vitamin C is safe and well tolerated by two different mouse strains. (A) Bodyweight of nod scid mice (NSG) bearing HCT116 derived tumors undergoing 3-days FMD alone or in combination with vitamin C (Vit C; 4 g/kg twice a day, i.p.). (B) Bodyweight of Balb/c mice bearing CT26.WT-derived tumors undergoing 3-days FMD alone or in combination with vitamin C (Vit C; 4 g/kg twice a day, i.p.). Bodyweight was recorded daily and it is indicated as percentage of weight at day 7.

FIG. 5. STS increases cellular ROS levels and enhances vitamin C pro-oxidant effect. HCT116 were grown in CTR or STS condition for 24 hours and then co-treated with vitamin C (Vit C; 1 mM) and the ROS-staining fluorescent probe CellROX Deep Red for 30 minutes. Median fluorescence intensity (MFI) was measured by flow cytometry (n≥3 biological replicates). Data are represented as mean±. Two-tailed, unpaired t-test was performed value (*p value≤0.05, ***p value≤0.001, ****p value≤0.0001).

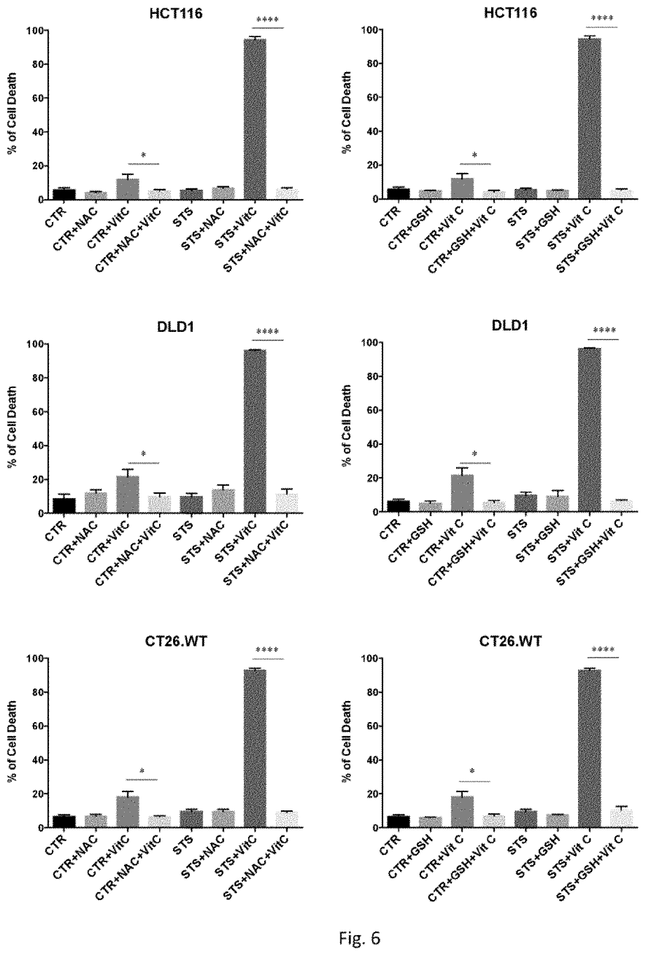

FIG. 6. GSH and NAC reverse STS+vitamin C toxicity in KRAS mutant CRC cells. HCT116, DLD1 and CT26.WT, were grown in CTR or STS condition for 24 hours. Then, cells were co-treated with the antioxidants glutathione (GSH; 5 mM) and glutathione precursor N-acetyl cysteine (NAC; 5 mM) and vitamin C (Vit C; 350 μM, for 24 hours). At 48 hours, viability was assessed by Muse Cell viability analyser (n≥3 biological replicates). Data are represented as mean±SEM. Two-tailed, unpaired t-test was performed value (*p value≤0.05, ****p value≤0.0001).

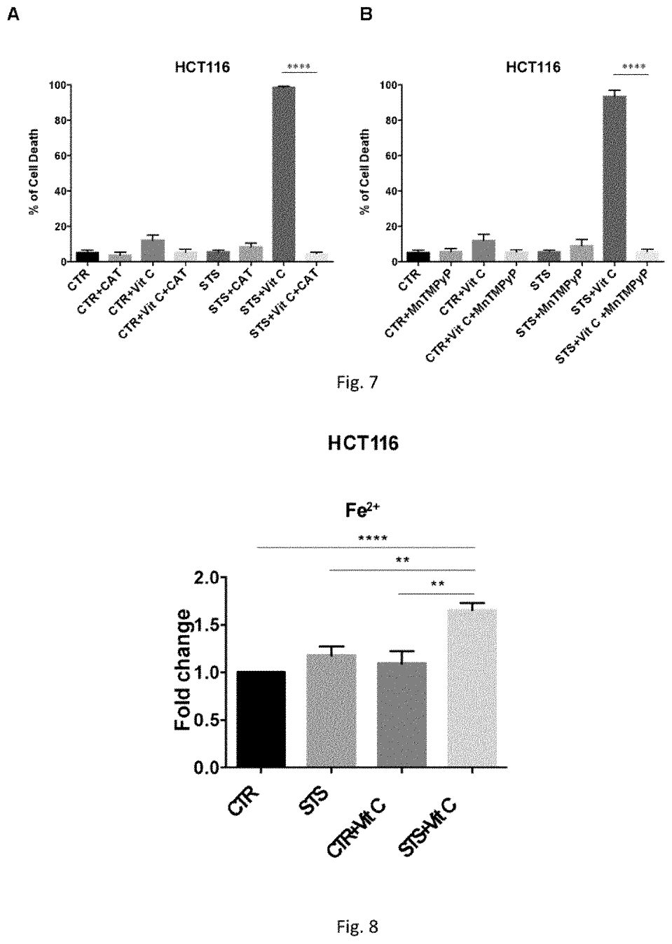

FIG. 7. Catalase and MnTMPyP reverse STS-mediated sensitization to vitamin C toxicity in HCT116 cells. HCT116 were grown in CTR or STS condition for 24 hours and treated with catalase (CAT; 50 U/ml) (A) or the superoxide dismutase (SOD)/catalase mimetic MnTMPyP (MnTMPyP; 50 μM) (B) prior to vitamin C (Vit C; 350 μM for 24 hours). At 48 hours, viability was assessed by Muse Cell viability analyser (n≥3 biological replicates). Data are represented as mean±SEM. Two-tailed, unpaired t-test was performed value (****p value≤0.0001).

FIG. 8. FMD and vitamin C combination treatment increases free iron (Fe′) level in HCT116 cells. HCT116 cells were grown in CTR or STS condition for 24 hours, followed by 3 hours of vitamin C treatment (Vit C; 350 μM). Then, cells were lysed and free iron (Fe²⁺) was measured by iron assay kit (n=5 biological replicates). Data are represented as mean±SEM. Two-tailed, unpaired t-test was performed (**p value≤0.01, ****p value≤0.0001).

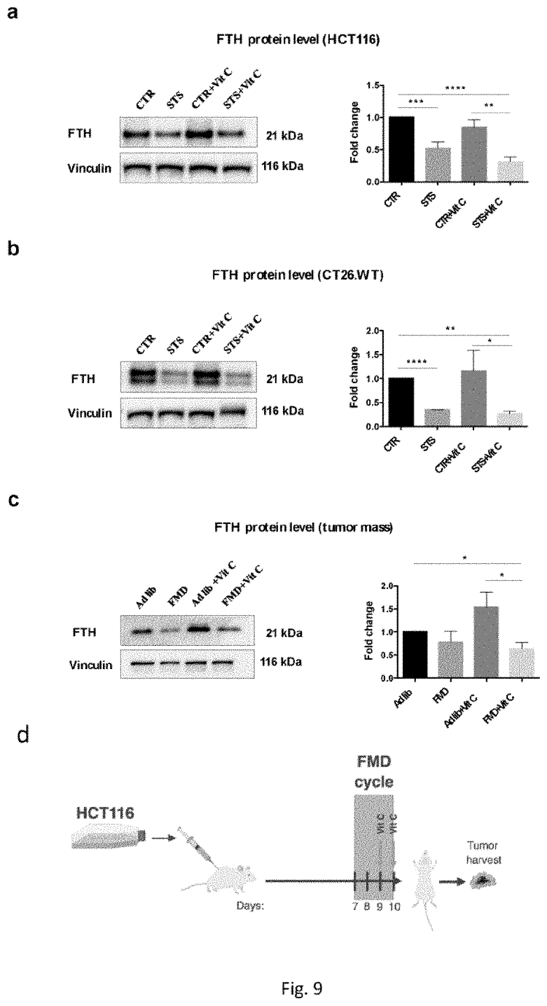

FIG. 9. STS and vitamin C combination treatment reduces FTH protein expression level. FTH protein expression level in HCT116 (a), CT26.WT (b) grown in CTR or STS condition for 24 hours, followed by 3 hours of vitamin C treatment (Vit C; 350 μM), were measured by western blotting (n≥5 biological replicates); FTH protein expression level of grafted HCT116 tumors (c) collected from mice fed ad libitum or undergoing FMD cycles (n≥3), were measured by western blotting; Vinculin as loading control. Vinculin as loading control. At the end of the cycle, mice were euthanized and tumors were excised and harvested for western blot analysis. Representative bands are shown (quantification on the right). Data are represented as mean±SEM. Two-tailed, unpaired t-test was performed (**p value≤0.01, ***p value≤0.001, ****p value≤0.0001). (d) Schematic representation of the in vivo experimental procedure. Mice were subcutaneously injected with HCT116 cells and when tumors were palpable (day 7) mice were subjected to FMD cycles, alone or in combination with high dose vitamin C (Vit C; 4 g/kg twice a day, i.p.).

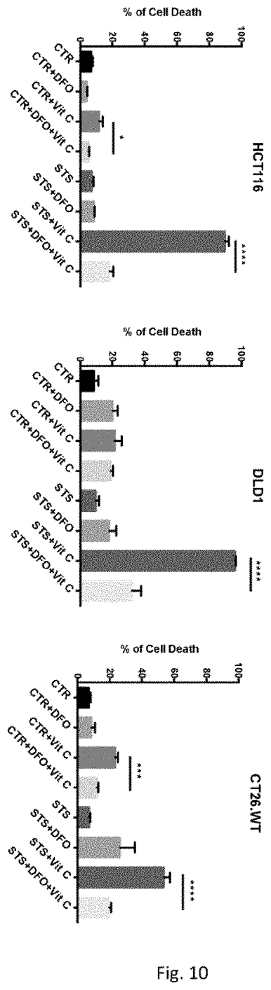

FIG. 10. DFO rescues STS and vitamin C cytotoxic effect. HCT116, DLD1 and CT26.WT were grown in CTR or STS condition for 24 hours and treated with desferrioxamine (DFO; 500 μM) for 6 hours (HCT116) or 12 hours (DLD1 and CT26.WT) before vitamin C treatment (Vit C; 350 μM). At 48 hours, viability was assessed by Muse Cell viability analyser (n≥3 biological replicates). Data are represented as mean±SEM. Two-tailed, unpaired t-test was performed value (*p value≤0.05, **p value≤0.01, ****p value≤0.0001).

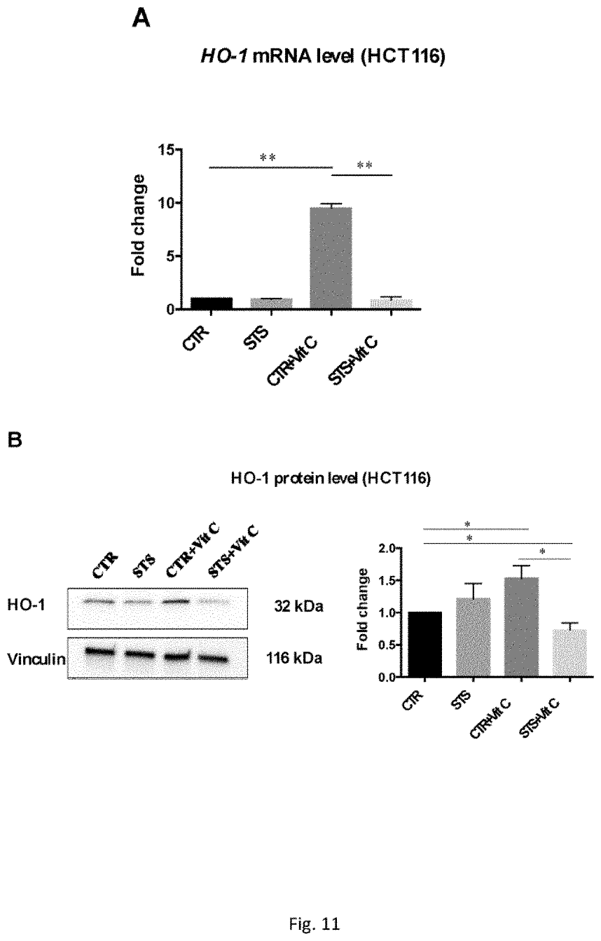

FIG. 11. Vitamin C up-regulates HO-1 expression level and STS reverts this effect in HCT116 cells. (A) HO-1 mRNA level in HCT116 grown in CTR or STS condition for 24 hours, followed by 3 hours of vitamin C treatment (Vit C; 350 μM), were analysed by qPCR. (B) HO-1 protein expression level in HCT116 grown in CTR or STS condition for 24 hours, followed by 3 hours of vitamin C treatment (Vit C; 350 μM), were measured by western blotting. Vinculin as loading control. Representative bands of at least three independent experiments are shown (quantification on the right). Data are represented as mean±SEM. Two-tailed, unpaired t-test was performed (*p value≤0.05, **p value≤0.01).

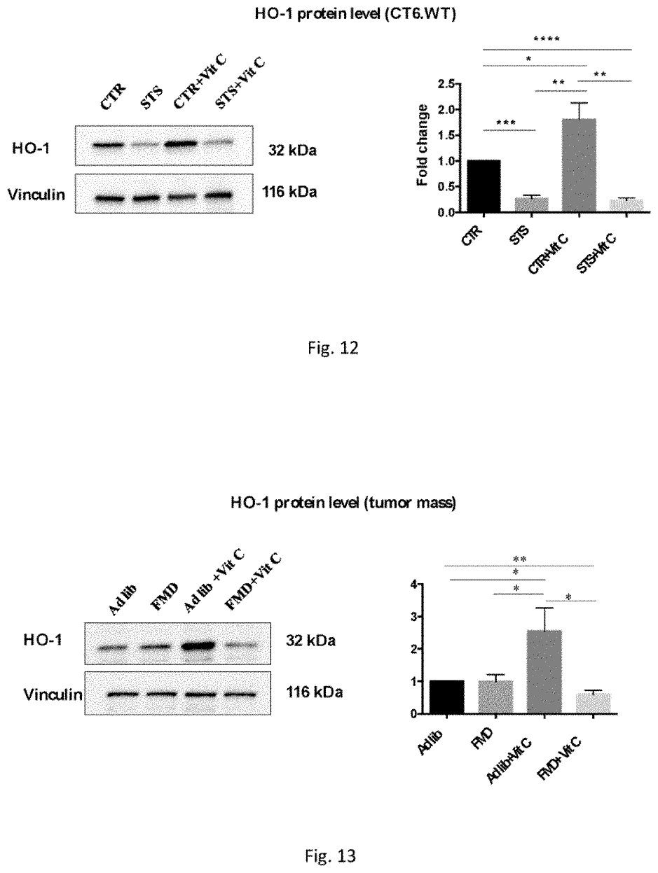

FIG. 12. Vitamin C up-regulates HO-1 expression level and STS reverts this effect in CT26.WT. HO-1 protein expression level in CT26.WT grown in CTR or STS condition for 24 hours, followed by 3 hours of vitamin C treatment (Vit C; 350 μM), were measured by western blotting. Vinculin as loading control. Representative bands are shown (quantification on the right). Data are represented as mean±SEM. Two-tailed, unpaired t-test was performed (*p value≤0.05, **p value≤0.01, ***p value≤0.001, ****p value≤0.0001).

FIG. 13. Vitamin C up-regulates HO-1 expression level and FMD reverts this effect in grafted tumors. HO-1 protein expression level of grafted HCT116 tumors collected from mice fed ad libitum or undergoing FMD cycles (n=5), were measured by western blotting. Vinculin as loading control. Representative bands are shown (quantification on the right). Data are represented as mean±SEM. Two-tailed, unpaired t-test was performed (*p value≤0.05, **p value≤0.01)

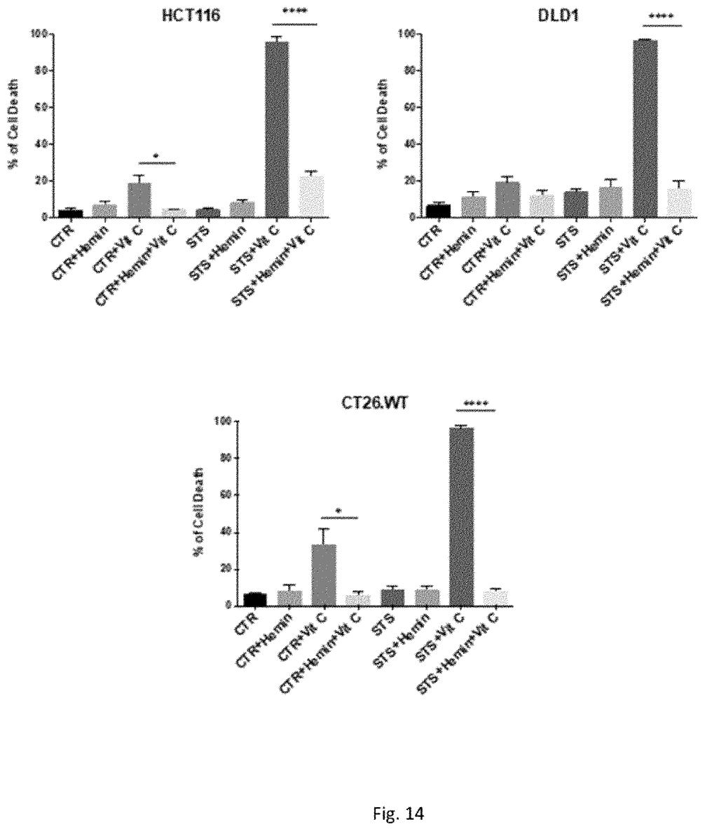

FIG. 14. HO-1 activation reverses FMD-mediated sensitization to vitamin C. HCT116, DLD1 and CT26.WT, were grown in CTR or STS condition for 24 hours. Cells were treated with hemin (20 μM) for 3 hours, before vitamin C treatment (Vit C; 350 μM, for 24 hours). At 48 hours, viability was assessed by Muse Cell viability analyser (n≥3 biological replicates). Data are represented as mean±SEM. Two-tailed, unpaired t-test was performed value (*p value≤0.05, ****p value≤0.0001).

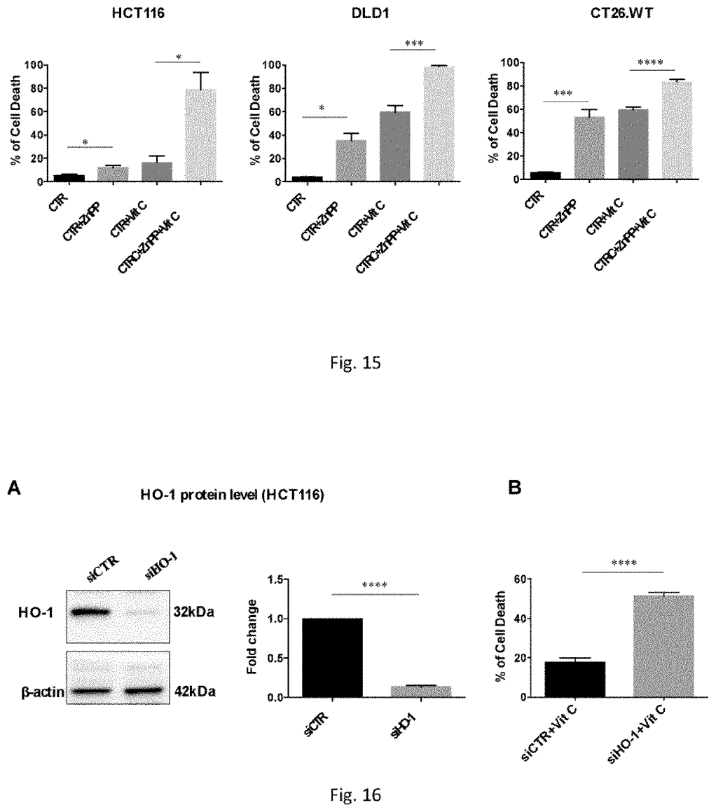

FIG. 15. Pharmacological HO-1 inhibition sensitizes cancer cells to vitamin C toxicity. HCT116, DLD1 and CT26.WT cell lines, were grown in CTR or STS condition for 24 hours. Cells were treated with the HO-1 inhibitor zinc protoporphyrin (ZnPP; 20 μM) for 3 hours, before vitamin C treatment (Vit C; 700 μM, for 24 hours). At 48 hours, viability was assessed by Muse Cell viability analyser (n≥3 biological replicates). Data are represented as mean±SEM. Two-tailed, unpaired t-test was performed value (*p value≤0.05, ***p value≤0.001, ****p value≤0.0001).

FIG. 16. HO-1 knockdown sensitizes cancer cells to vitamin C toxicity. (A) HCT116 cells were transfected with siHO-1 or siCTR and HO-1 knockdown was assessed by western blotting. (3-actin as loading control. Representative bands are shown (quantification on the right). (B) HCTT16, in CTR condition, were transfected with control siRNAs (siCTR) or a pool of siRNAs against HO-1 (siHO-1) and, after 24 hours, treated with vitamin C (Vit C; 700 μM) for 24 hours. Viability was assessed by Muse Cell viability analyser (n≥3 biological replicates). Data are represented as mean±SEM. Two-tailed, unpaired t-test was performed value (****p value≤0.0001).

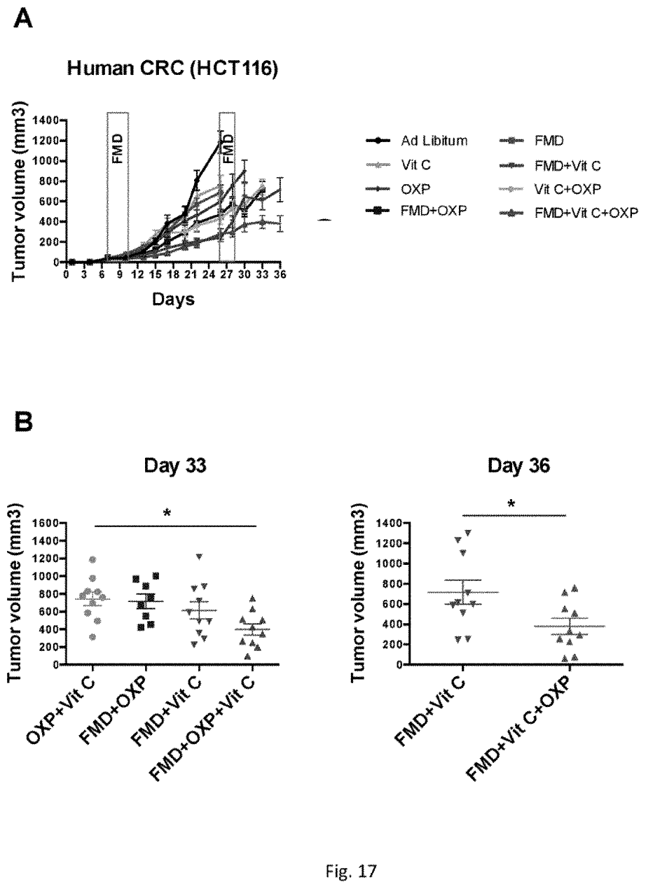

FIG. 17. FMD, vitamin C, Oxaliplatin triple treatment is effective in delaying tumor progression in HCT116 xenograft mouse model. 12-weeks old female NOD scid gamma (NSG) mice were subcutaneously injected with HCT116 cells. Mice were fed ad libitum or subjected to FMD cycles, and treated with high dose vitamin C (Vit C; 4 g/kg twice a day, i.p.) or saline, Oxaliplatin (OXP; 10 mg/kg i.p. once every 15 days) or vehicle, as single agents or as combination. Tumor volumes at multiple time points (A) and before mice were euthanized (Days 33 and 36) (B) are reported (n=8-10). Data are represented as mean±SEM. One-way Anova (Tukey's post-analysis test) and two-tailed unpaired t-test (for day 36) were performed (*p value≤0.05).

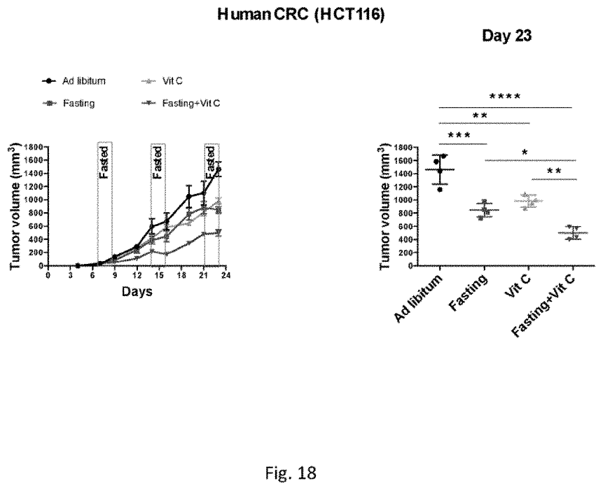

FIG. 18. Fasting cycles enhances vitamin C toxicity in reducing tumor progression in HCT116 xenograft. 8-weeks old female NOD scid gamma (NSG) mice were subcutaneously injected with HCT116 cells and subjected to 3 cycles of fasting (2 days, water only) alone or in combination with high dose vitamin C (Vit C; 4 g/kg twice a day, i.p.). Tumor volumes at multiple time points (left panel) and before mice were euthanized (right panel) are reported (n=4 per group). Data are represented as mean±SEM. One-way Anova (Tukey's post-analysis test) was performed (*p value≤0.05, **p value≤0.01, ***p value≤0.001, ****p value≤0.0001).

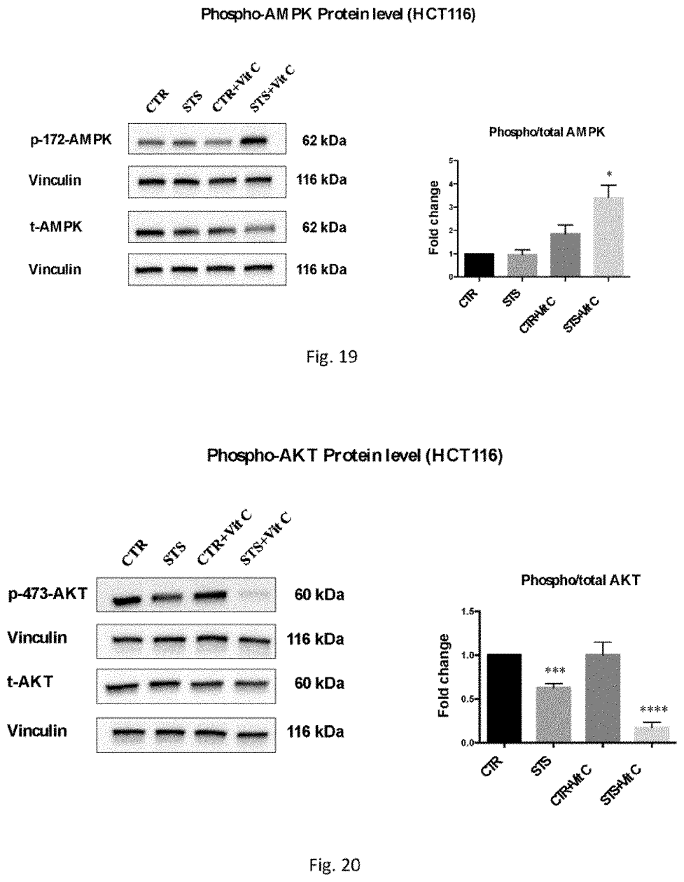

FIG. 19. STS and Vitamin C combo treatment up-regulates AMPK phosphorylation level. HCT116 cells were grown in CTR or STS condition for 24 hours, followed by 3 hours of vitamin C treatment (Vit C; 350 μM). AMPK and phospho-AMPK (threonine 172) protein expression levels were measured by western blotting. Vinculin as loading control. Representative bands of three biological replicates are shown (quantification on the right). Data are represented as mean±SEM. Two-tailed, unpaired t-test was performed (*p value≤0.05).

FIG. 20. STS and Vitamin C combo treatment down-regulates AKT phosphorylation level. HCT116 cells were grown in CTR or STS condition for 24 hours, followed by 3 hours of vitamin C treatment (Vit C; 350 μM). AKT and phospho-AKT (serine 473) protein expression level were measured by western blotting. Vinculin as loading control. Representative bands of three biological replicates are shown (quantification on the right). Data are represented as mean±SEM. Two-tailed, unpaired t-test was performed, (***p value≤0.001, ****p value≤0.0001).

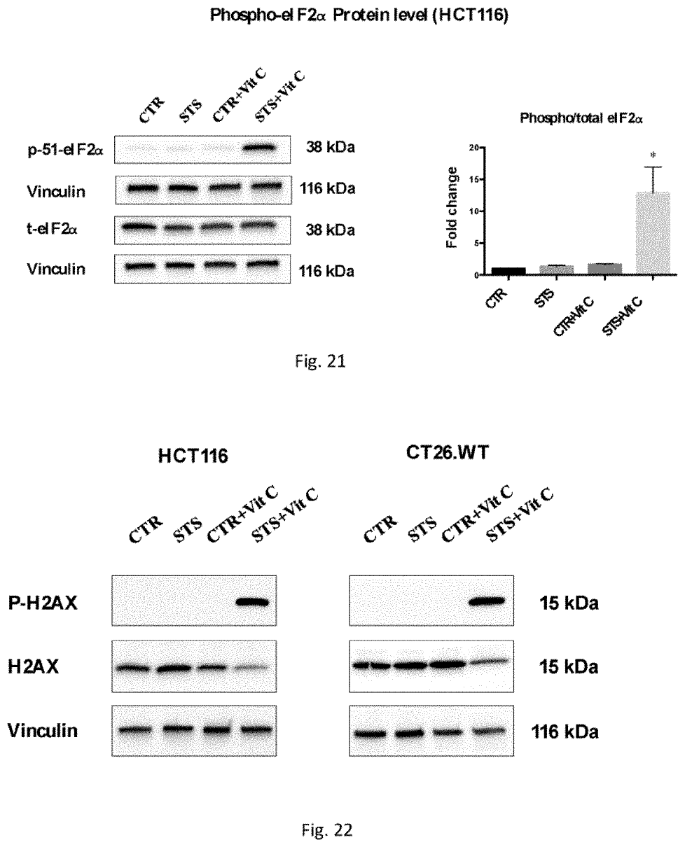

FIG. 21. STS and Vitamin C combo treatment up-regulates eIF2a phosphorylation level. HCT116 cells were grown in CTR or STS condition for 24 hours, followed by 3 hours of vitamin C treatment (Vit C; 350 μM). eIF2a and phospho-eIF2a (serine 51) protein expression level were measured by western blotting. Representative bands of three biological replicates are shown (quantification on the right). Data are represented as mean±SEM. Two-tailed, unpaired Student's t-test was performed (*p value≤0.05, **p value≤0.01).

FIG. 22. STS and vitamin C co-treatment induces phosphorylation of histone H2AX. Phosphorylated histone H2AX and total level of H2AX in HCT116 and CT26.WT grown in CTR or STS condition for 24 hours, followed by 3 hours of vitamin C treatment (Vit C; 350 μM), were measured by western blotting, representative bands of three independent experiments are shown. Vinculin as loading control.

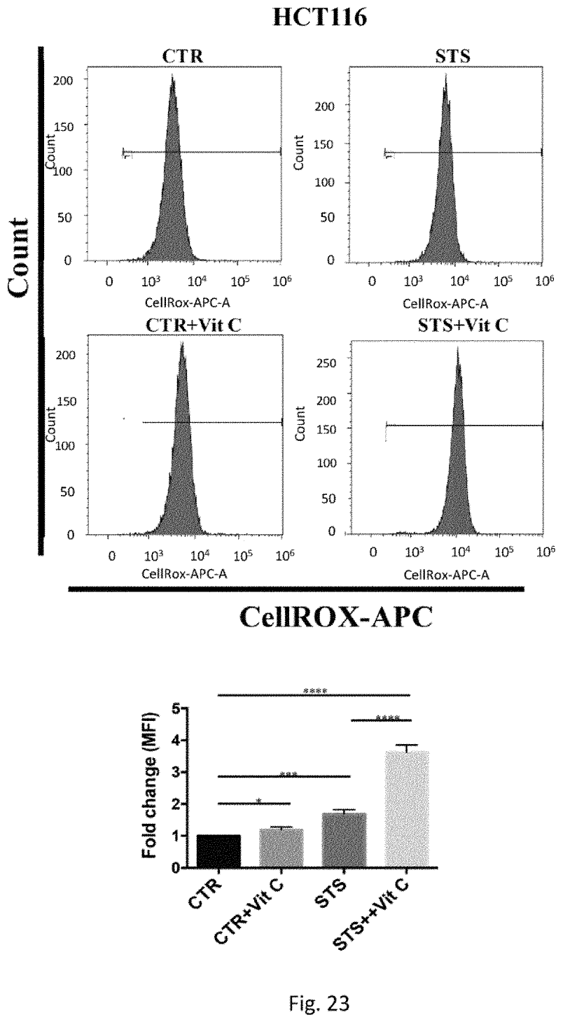

FIG. 23. STS increases cellular ROS levels and enhances vitamin C pro-oxidant effect. HCT116 were grown in CTR or STS condition for 24 hours and then co-treated with vitamin C (Vit C; 1 mM) and the ROS-staining fluorescent probe CellROX Deep Red for 30 minutes. Median fluorescence intensity (MFI) was measured by flow cytometry (n≥3 biological replicates). Representative histogram (left panel) and quantification (right panel) were shown. Data are represented as mean±SEM. Two-tailed, unpaired t-test was performed value (*p value≤0.05, ***p value≤0.001, ****p value≤0.0001).

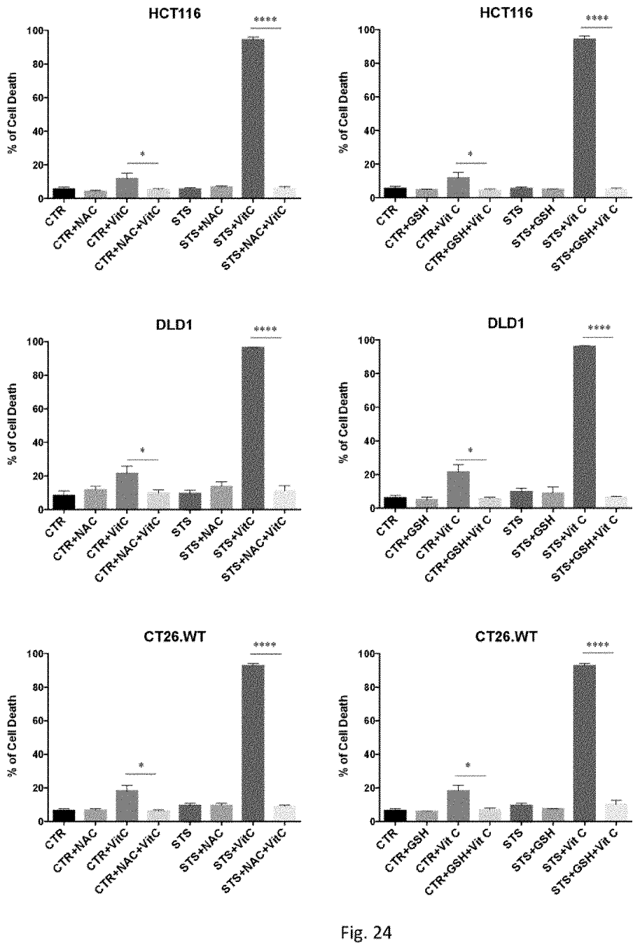

FIG. 24. GSH and NAC reverse STS+vitamin C toxicity in KRAS mutant CRC cells. HCT116, DLD1 and CT26.WT, were grown in CTR or STS condition for 24 hours. Then, cells were co-treated with the antioxidants glutathione (GSH; 5 mM) and glutathione precursor N-acetyl cysteine (NAC; 5 mM) and vitamin C (Vit C; 350 μM, for 24 hours). At 48 hours, viability was assessed by Muse Cell viability analyser (n≥3 biological replicates). Data are represented as mean±SEM. Two-tailed, unpaired t-test was performed value (*p value≤0.05, ****p value≤0.0001).

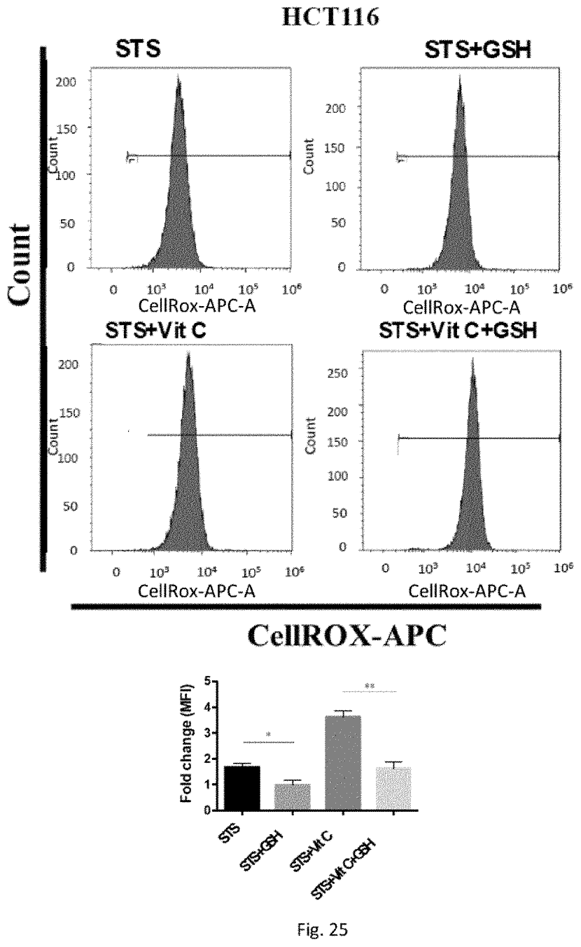

FIG. 25. GSH reverses STS and STS+vitamin C-mediated increase in cellular oxidative stress in HCT116. HCT116 were grown in CTR or STS condition for 24 hours and then treated with GSH one hour prior to vitamin C treatment. Next, cells were cotreated with vitamin C (Vit C; 1 mM) and the ROS-staining fluorescent probe CellROX Deep Red for 30 minutes. Median fluorescence intensity (MFI) was measured by flow cytometry (n≥3 biological replicates). Representative histogram on the left, quantification on the right. Data are represented as mean±SEM. Two-tailed, unpaired t-test was performed value (*p value≤0.05, ***p value≤0.001).

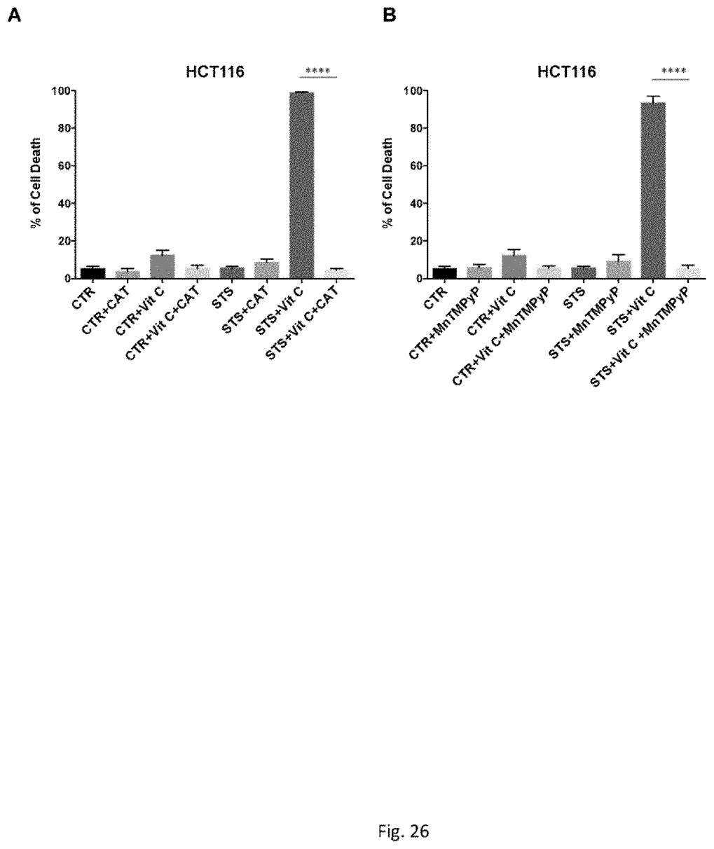

FIG. 26. Catalase and MnTMPyP reverse STS-mediated sensitization to vitamin C toxicity in HCT116 cells. HCT116 were grown in CTR or STS condition for 24 hours and treated with catalase (CAT; 50 U/ml) (A) or the superoxide dismutase (SOD)/catalase mimetic MnTMPyP (MnTMPyP; 50 μM) (B) prior to vitamin C (Vit C; 350 μM for 24 hours). At 48 hours, viability was assessed by Muse Cell viability analyser (n≥3 biological replicates). Data are represented as mean±SEM. Two-tailed, unpaired t-test was performed value (****p value≤0.0001).

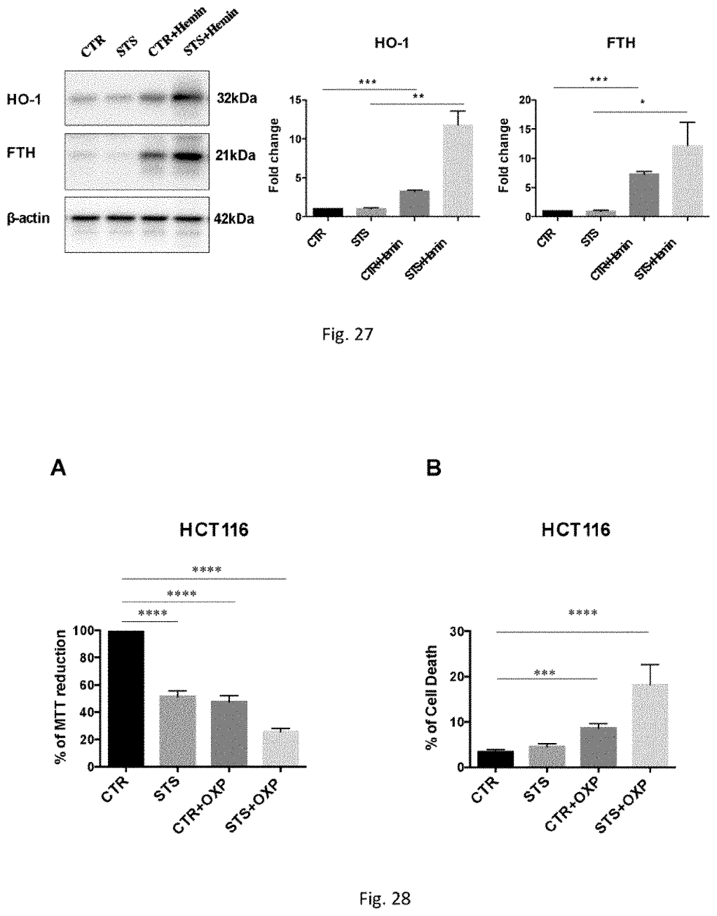

FIG. 27. Hemin up-regulates HO-1 and FTH protein expression level. HCT116 were grown in CTR or STS condition for a total of 24 hours. At 12 hours, cells were treated with the HO-1 activator hemin (20 μM) for the next 12 hours. HO-1 and FTH protein expression level were measured by western blotting; (3-actin as loading control. Representative bands of three independent experiments are shown (quantifications on the right). Data are represented as mean±SEM. Two-tailed, unpaired t-test was performed (*p value≤0.05, **p value≤0.01, ***p value≤0.001).

FIG. 28. STS sensitizes HCT116 to oxaliplatin toxicity. HCT116 were grown in CTR or STS conditions for a total of 48 hours. At 24 hours, cells were treated with oxaliplatin (OXP; 40 μM) or vehicle for further 24 hours. At the end of the experiment, cell viability was assessed by MTT reduction (A), whereas percentage of cell death was assessed by erythrosine B exclusion assay (B) (n≥3 biological replicates). Data are represented as mean±SEM (two-tailed unpaired t-test; ***p value≤0.001, ****p value≤0.0001).

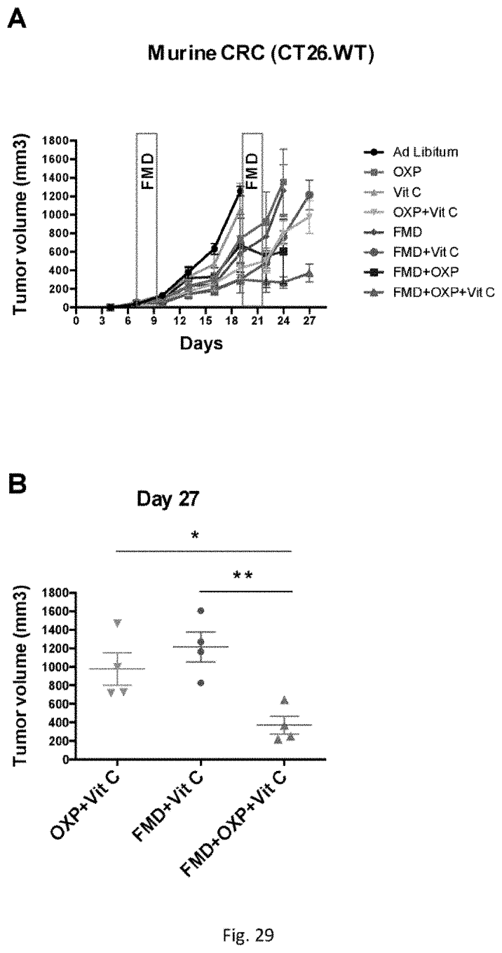

FIG. 29. FMD, vitamin C, Oxaliplatin triple treatment is effective in delaying tumor progression in CT26.WT allograft mouse model. 8-weeks old female Balb c/OlaHsd mice were subcutaneously injected with CT26.WT cells. Mice were fed ad libitum or subjected to FMD cycles, and treated with high dose vitamin C (Vit C; 4 g/kg twice a day, i.p.) or saline, oxaliplatin (OXP; 10 mg/kg i.p. once every 10 days) or vehicle, as single agents or as combination. Tumor volumes at multiple time points (A) and before mice were euthanized (Day 27) (B) are reported (n=3-4 per group). Data are represented as mean±SEM. One-way Anova (Tukey's post-analysis test) was performed (*p value≤0.05, **p value≤0.01).

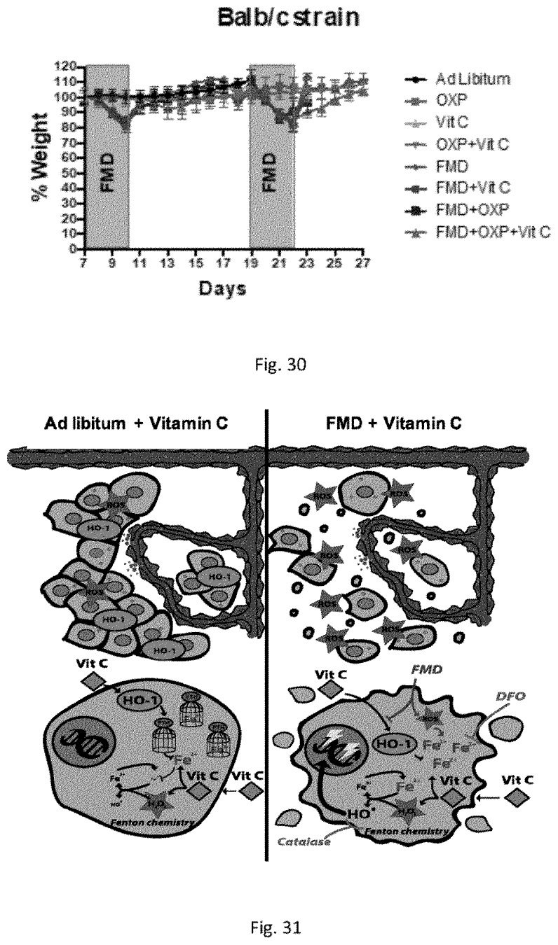

FIG. 30. FMD, vitamin C, Oxaliplatin triple treatment is safe and well tolerated. Weight of Balb/cOlaHsd mice bearing CT26.WT-derived tumors underwent 3-days FMD alone or in combination with vitamin C (Vit C; 4 g/kg twice a day, i.p.) and Oxaliplatin (OXP; 10 mg/kg i.p. once every 10 days). Bodyweight was recorded daily and it is indicated as percentage of weight at day 7.

FIG. 31. Working model of FMD-mediated sensitization to vitamin C. Vitamin C oxidation generates H₂O₂ and, by reacting with Fe²⁺ (Fenton chemistry), produces HO□. Vitamin C-mediated up-regulation of HO-1 induces FTH, thus limiting LIP and consequently the pro-oxidant chemistry responsible for vitamin C toxicity (left side). FMD is able to revert the HO-1 up-regulation induced by vitamin C. By doing this, FMD expands Fe²⁺ pool possibly through FTH down-regulation. The increase in Fe²⁺, together with the FMD-induced boost in ROS levels possibly exacerbate Fenton chemistry leading to DNA damage (yellow bolts) and cell death. Catalase, by scavenging H₂O₂, and DFO, by chelating iron, inhibit Fenton reaction and prevent cell death (right side).

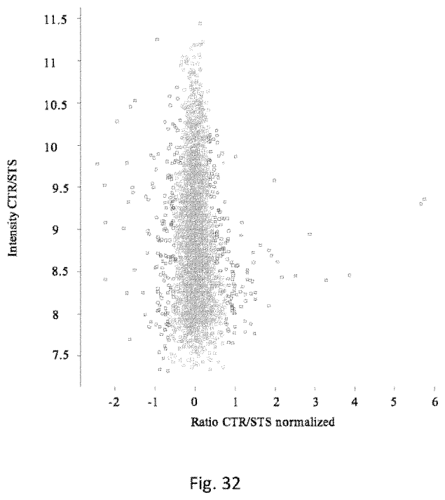

FIG. 32. SILAC analysis of proteome alteration of KRAS mutant CRC cells upon STS. HCT116 were grown in SILAC DMEM media supplemented with 1 g/l glucose, 10% dialysed serum (CTR) and “heavy” amino acids arginine and lysine, whereas cells that have incorporated “light” amino acids, were then grown in SILAC DMEM media supplemented with 0.5 g/l glucose and 1% dialysed serum (STS). Statistically significative proteins that differ in intensity in CTR versus STS condition are indicate by red boxes. Unaffected proteins are in grey boxes. Significance B outlier test was applied (p<0.05).

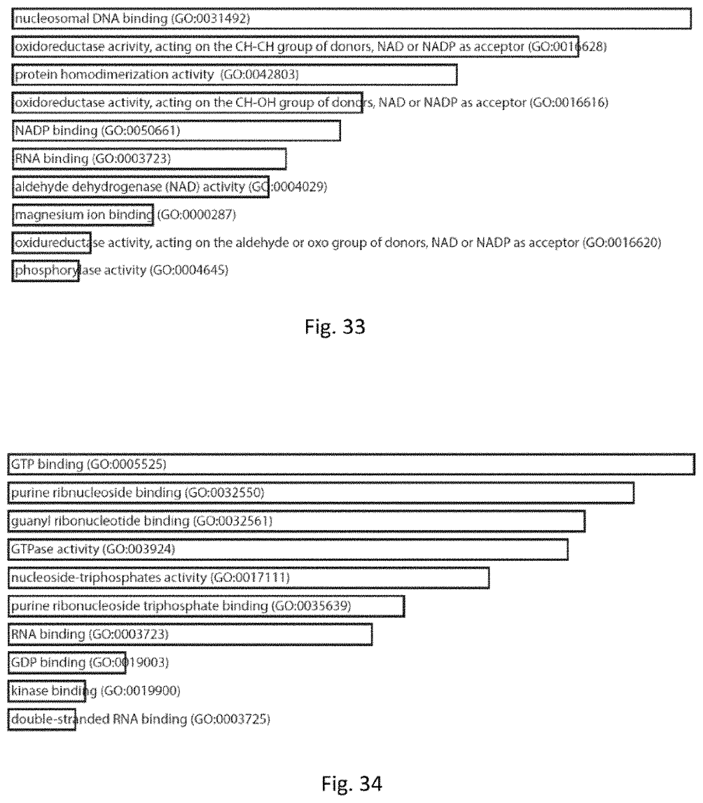

FIG. 33. Gene Ontology (GO) enrichment analysis of proteins up-regulated upon STS. Gene Ontology (GO) enrichment analysis with Enrichr of the 167 proteins up-regulated in STS, compared to CTR condition, in KRAS mutant HCT116 cells. Bar graph show the top 10 enriched Molecular function GO gene-set library sorted by p-value ranking. The length of bars indicates the relative significance (Fisher Exact test; adjusted p-value: from 0.001045 [GO:0031492] to 0.03018 [GO:00046451].

FIG. 34. Gene Ontology (GO) enrichment analysis of proteins down-regulated upon STS. Gene Ontology (GO) enrichment analysis with Enrichr of the 141 proteins down-regulated in STS, compared to CTR condition, in KRAS mutant HCT116 cells. Bar graph show the top 10 enriched Molecular function GO gene-set library sorted by p-value ranking. The length of bars indicates the relative significance (Fisher Exact test; adjusted p-value: from 0.01487 [GO:0005525] to 0.02052 [GO:00037251].

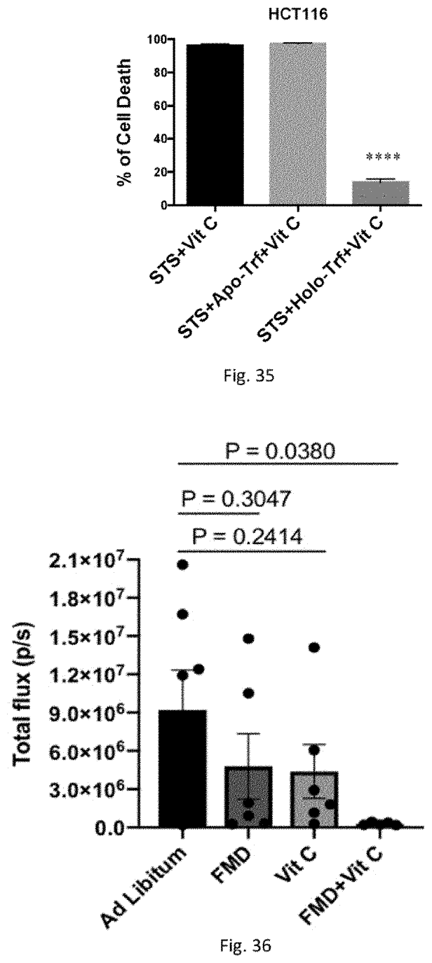

FIG. 35. Holo-transferrin, but not apo-transferrin, reverses STS-mediated sensitization to vitamin C toxicity. HCT116 were grown in STS medium supplemented with apo-transferrin (Apo-Trf; 25 ng/ml) or holo-transferrin (Holo-Trf; 25 ng/ml) before vitamin C (Vit C; 350 μM) exposure. At 48 hours, viability was assessed by Muse Cell viability analyser. Data are represented as mean±SEM (two-tailed unpaired t-test; ****p value≤0.0001).

FIG. 36. Fasting Mimicking diet (FMD) enhances vitamin C anticancer activity in KRAS-mutant tumors.

(f) CT26-luc-derived orthotopic model (n=5-7 mice/group). 2.5×10⁴ CT26-luc cells (SC061-LG GenTarget Inc), suspended in saline, were injected submucosally into the distal, posterior rectum. Seven days later, mice were randomly divided in the different experimental groups. Mice were fed ad libitum or underwent 2 FMD cycles and were daily treated with Vitamin C or vehicle. Twenty-one days post injections mouse imaging was performed using the Xenogen IVIS-200 System. Total photon effluxes over tumor regions were measured. Data are represented as mean±SEM.

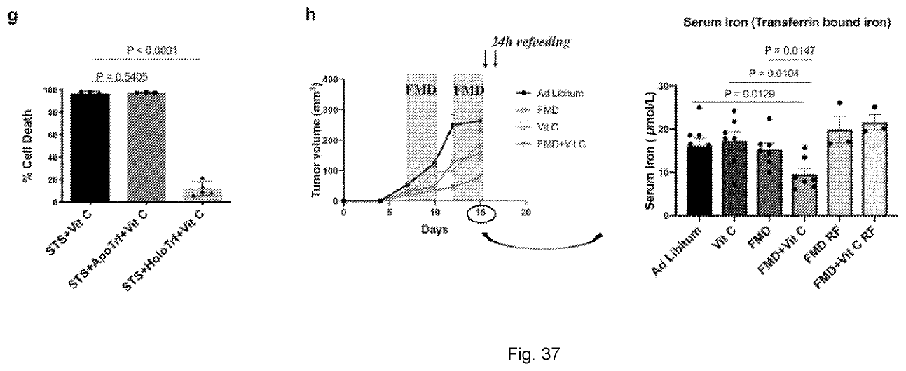

FIG. 37. HO-1 modulation and iron-bound transferrin are the key players in FMD-dependent sensitization to Vitamin C. (g) HCT116 cells were grown in STS medium and treated with Vitamin C, in presence or absence of apo-transferrin (iron-free form) or holo-transferrin (iron-bound form) and viability was assessed (n=3-5 biological replicates). (h) HCT116-xenograft mice were randomly divided in the different experimental groups. Mice were fed ad libitum or underwent 2 FMD cycles and were daily treated with Vitamin C or vehicle. Mouse blood was collected from the heart of mice sacrificed at the end of 2^{nd} FMD cycle and 24 hours post-refeeding.

Transferrin bound iron in mouse serum was measured (n=3-10 mice per group). All data are represented as mean±SEM.

### DETAILED DESCRIPTION OF THE INVENTION

### Example 1

Fasting-Mimicking Diet Based Non-Toxic Interventions as Novel Therapy for Kras Mutant Cancer

The inventors discovered that FMD potentiate the pro-oxidant action of vitamin C in KRAS mutant tumors, representing a novel opportunity for cancer patients.

KRAS Mutant CRC Cells are Sensitive to Pharmacological Vitamin C Toxicity

The inventors used as an in vitro model system the human KRAS mutant (G13D) CRC cell line HCT116 in order to identify an intervention that could cause a major potentiation of the toxicity of vitamin C to cancer cells without causing toxicity to normal cells.

To test vitamin C efficacy, HCT116 cells were grown in complete medium, which mimic physiological level of glucose and serum (1 g/L glucose, 10% serum), indicated as control (CTR) medium. When cells reach 40% confluency, they were treated with pharmacological dose of vitamin C (≥0.3 mM) for 24 hours. As shown in FIG. 1, in my experimental condition, high-dose vitamin C was able to induce cell death, as reported in previous published findings (Yun et al., 2015) (FIG. 1-1).

In Vitro Short-Term Starvation (STS) Sensitizes KRAS Mutant Cancer Cells to Pharmacological Vitamin C Toxicity

The inventors investigated whether our recently developed FMD diet could potentiate the anti-cancer effect of vitamin C in these highly aggressive tumors. The efficacy of FMD was assessed in vitro through a low-glucose and low-serum cell-growing medium, referred as short-term starvation condition (STS), which mimics the glucose and growth factor reduction mediated by fasting/FMD in in vivo physiological setting. Whereas complete medium mimics physiological level of glucose and serum (1 g/L glucose, 10% serum), indicated as control (CTR) medium.

To this purpose, human (HCT116, DLD-1) and murine (CT26.WT) KRAS mutant CRC cell lines and KRAS mutant lung cancer (H23, H727) and pancreatic cancer (PANC1) cell lines, grown in CTR or STS condition, were treated with pharmacological dose vitamin C (≥0.3 mM) for 24 hours. In our experimental condition, the inventors found that KRAS mutant cancer cells showed a modest sensitivity to vitamin C However, when cells were grown in STS condition, 24 hours before and during treatment, vitamin C-mediated toxicity was strongly enhanced (FIG. 1a). Conversely, KRAS wild type cell lines derived from colorectal cancer (SW48, HT29), prostate cancer (PC-3), and ovarian cancer (COV362) or normal colon cell line (CCD841CoN) and normal fibroblasts (BJ) were not sensitive to vitamin C treatment, either as single agent or in combination with STS condition (FIG. 1b).

FMD Cycles Delay Tumor Progression and Enhance Vitamin C Anti-Cancer Effect in KRAS Mutant CRC Mouse Models

Supported by their in vitro results, the inventors investigated whether FMD cycles could enhance vitamin C toxicity also in mouse models of KRAS mutant colorectal cancer. FMD is a low calories, protein and sugar but high unsaturated fat diet, which is able, as fasting, to delay tumor progression and sensitize cancer cells to chemotherapy (Brandhorst et al., 2015; Di Biase et al., 2016).

For this purpose, nod scid mice (NSG) bearing HCT116 subcutaneous tumors, were fed ad libitum with standard rodent diet or FMD for three days every week.

It has been already described that the parenteral administration mediated by intraperitoneal injection, allows to reach high-dose vitamin C (3 mM), which is necessary to obtain its anti-cancer effect (Chen et al., 2007; Chen et al., 2008). Thus, mice were treated with saline or high dose vitamin C (4 g/kg) intraperitoneally (i.p.). twice a day, every day, as previously described (Chen et al., 2007; Chen et al., 2008).

Interestingly, the inventors found that, 3-days of a FMD every week, delayed tumor progression to the same extent as high-dose vitamin C in HCT116 tumor bearing mice (FIG. 2). Notably, three cycles of FMD combined with daily treatment of pharmacological vitamin C, showed the best therapeutic outcome in reducing tumor progression by 3-fold compared to mice fed ad libitum, and by 2-fold compared to mice undergoing FMD or vitamin C (FIG. 2).

Next, the inventors assessed the effect of FMD and vitamin C combo treatment also in a syngeneic mouse model of KRAS-driven CRC. In mice bearing CT26.WT allograft, FMD and vitamin C combinatorial treatment showed a higher efficacy than FMD or vitamin C as single intervention, by reducing tumor volume by 6-fold compared to mice fed ad libitum, 2.6-fold compared to mice undergoing FMD and 3.4-fold compared to mice receiving vitamin C as single treatment (FIG. 3).

Importantly, the finding that FMD and vitamin C combinatorial therapy is effective in both immunocompromised and immunocompetent mouse models, suggests that at least part of the observed effect is not dependent on the immune system, but it involves a tumor specific component.

Furthermore, FMD and vitamin C combinatorial treatment resulted to be safe and well tolerated in both mouse strains, as indicated by mouse bodyweight (FIG. 4). During each FMD cycle, mice did not lose more than 20% of their initial weight, which is immediately recovered upon refeeding (FIG. 4). Cotreatment with vitamin C did not enhance FMD-dependent bodyweight loss and did not impair its recovery after each FMD cycle (FIG. 4).

FMD and Vitamin C Combo Treatment Induces Oxidative Stress

The inventors previously shown that fasting sensitizes different types of cancer cells to chemotherapy in part by increasing reactive oxygen species (ROS) (Lee et al, 2012). The inventors tested whether FMD together with vitamin C enhances ROS production in KRAS mutant colorectal cancer (CRC) cells.

ROS, which include H₂O₂ and superoxide (O₂.⁻), are generated as by-products of normal metabolisms. However, excessive ROS production, known as oxidative stress, have detrimental effects on cellular biomolecules, including DNA, lipids and proteins (Reczek and Chandel, 2017).

To explore this hypothesis, ROS levels were measured as indicated by the oxidation of the fluorogenic probe CellROX, in HCT116 cells grown in CTR or STS condition. Present results indicate that STS as single intervention was able to increase ROS levels, as well as vitamin C. However, the combinatorial treatment strongly exacerbated cellular oxidative state by 3.6-fold (FIG. 5).

To directly test whether high ROS level is a causative or a secondary event in STS-induced sensitization to vitamin C, KRAS mutant CRC cells (HCT116, DLD1, CT26.WT) grown in STS condition and exposed to vitamin C, were co-treated with glutathione (GSH), which is the major cellular antioxidant, as well as with the cell-permeable reducing agent and glutathione precursor N-acetyl cysteine (NAC). Both agents rescued vitamin C cytotoxic effect in CTR medium. Interestingly, GSH and NAC were also able to revert the massive cell death induction mediated by STS and vitamin C cotreatment, suggesting that STS may act by exacerbating the pro-oxidant action of vitamin C (FIG. 6).

Next, to further assess whether oxidative stress is the causative event in mediating STS+vitamin C cytotoxicity, the inventors tested the effect of exogenous membrane-impermeable catalase.

Present data show that exogenous catalase exposure prior vitamin C treatment, was able to revert STS-mediated sensitization to vitamin C (FIG. 7 A). In addition, the treatment with the membrane-permeable superoxide dismutase (SOD)/catalase mimetic MnTMPyP, which has been reported to scavenge both superoxide anions and also hydrogen peroxide, before vitamin C exposure, inhibited cell death upon combinatorial treatment (FIG. 7 B). Collectively present data strongly indicate that increased ROS levels are the causative factors responsible for STS-mediated sensitization to vitamin C anti-cancer effect.

FMD and Vitamin C Combo Treatment Increases Free Iron Pool

A large body of studies shows that the mechanism of vitamin C anti-cancer effect is mediated by hydrogen peroxide production (Chen et al., 2008; Schoenfeld et al., 2017). Particularly, its toxicity is dependent on metal ion redox chemistry, which allows the generation of the hydroxyl radical, through the Fenton reaction (Chen et al., 2008; Schoenfeld et al., 2017).

Schoenfeld and colleagues recently reported that ROS-induced disruption of iron metabolism, significantly contributes to vitamin C toxicity in tumors, by increasing the redox-active labile iron pool involved in the Fenton reaction (Schoenfeld et al., 2017).

Given the role of FMD and vitamin C in increasing ROS levels, the inventors investigated whether the oxidative stress enhancement could correlate with an increase in labile ferrous iron pool. Interestingly, the inventors found that, in HCT116 cells, ferrous ion (Fe²⁺) level was significantly higher upon STS and vitamin C combo treatment compared to all other conditions (FIG. 8), thus suggesting a potential role of iron in mediating FMD-sensitization effect to vitamin C treatment.

Ferritin, the main protein involved in iron binding and storage, plays a central role in the modulation of the intracellular labile iron pool (LIP) (Torti and Torti, 2013; Kakhlon et al., 2009; Yang et al., 2009). Particularly, ferritin down-regulation has been shown to be responsible for LIP increase in KRAS-mutant cancer cells (Kakhlon et al., 2009; Yang et al., 2008). Thus, the inventors investigated whether ferritin could be involved in the STS-mediated increase in ferrous ion observed upon combinatorial treatment. For this purpose, the inventors evaluated the levels of the heavy subunit of ferritin (FTH), which is responsible for the iron storage through its ferroxidase activity (Torti and Torti, 2013). The inventors found that STS down-regulated FTH protein expression in human HCT116 and in murine CT26.WT KRAS-mutant cells (FIGS. 9 a, b). The results obtained in vitro were also confirmed in vivo, where the inventors found that FMD cycles down-regulated FTH protein expression level also in HCT116 xenografts upon FMD cycles (FIG. 9 c).

To assess whether the alteration in cellular iron content is responsible for STS-mediated sensitization to vitamin C, HCT116 cells grown in STS conditions were treated with the iron chelator desferrioxamine (DFO) before vitamin C exposure. To specifically evaluate the intracellular effect of DFO (in chelating iron) and to avoid possible interactions with chemical components in the growth medium, cells were washed before vitamin C treatment. Consistent with presnthypothesis, DFO pre-treatment rescued vitamin C-induced cell cytotoxicity (FIG. 10), thus suggesting that the increase in intracellular free iron mediated by FMD/STS is, at least in part, responsible for the enhanced vitamin C oxidation and toxicity.

Vitamin C Up-Regulates Heme Oxygenase-1 Expression Level while FMD Reverts this Effect

Heme oxygenase-1 is a stress-inducible protein which plays a key role in mediating anti-oxidant and anti-apoptotic response in different tumor types, making its induction a mechanism of therapy resistance (Was et al., 2010; Busserolles et al., 2006; Liu et al, 2004; Kim et al., 2008; Berberat et al., 2005).

Notably, inventors' previous works have already shown that FMD sensitizes breast cancer to chemotherapy in part by down-regulating HO-1, supporting a possible role of this stress-inducible protein in mediating FMD beneficial effects (Di Biase et al., 2016).

The inventors evaluated HO-1 expression level in CTR and STS media and in presence or absence of vitamin C treatment. Interestingly, the inventors found that vitamin C, as single intervention, significantly up-regulated HO-1 both at transcriptional and protein level in HCT116 cell line, while STS was able to revert this effect (FIG. 11). Similar results were also obtained in the murine CT26.WT cell line (FIG. 12).

Importantly, also in vivo in grafted tumor masses derived from HCT116 cells, FMD cycles reversed the HO-1 up-regulation mediated by vitamin C treatment (FIG. 13).

Collectively, these findings point out the importance of HO-1 in FMD-sensitization of vitamin C cytotoxic effect.

The effect of FMD in reverting HO-1 up-regulation mediated by vitamin C, prompted the inventors to investigate whether modulation in HO-1 expression level could alter cancer cell-sensitivity to pharmacological vitamin C treatment. To this purpose, human and murine KRAS mutant CRC cell lines (HCT116, DLD1, CT26.WT), grown in CTR or STS condition, were exposed to the HO-1 activator hemin. Notably, the inventors found that, HO-1 activation was able to reverse cancer cell death induced by both vitamin C alone or in combination with STS (FIG. 14).

Next, the inventors investigated whether HO-1 inhibition could sensitize cancer cells to the toxic effect of vitamin C in control condition. The inventors found that the HO-1 inhibitor zinc protoporphyrin (ZnPP) was able to make KRAS mutant CRC cells more susceptible to vitamin C toxicity in control medium (FIG. 15).

Accordingly, also HO-1 knockdown in HCT116 cells, increased cell death rate upon vitamin C treatment in nutrient-rich condition (FIG. 16), supporting the key role of HO-1 in determining cancer cell sensitivity to vitamin C.

FMD and Vitamin C Combo Treatment Potentiates Oxaliplatin Efficacy in a Mouse Model of KRAS Mutant CRC

Several studies described the tolerability of high dose vitamin C as adjuvant treatment during chemotherapy (Monti et al., 2012; Ma et al., 2014; Schoenfeld et al., 2017). In addition, the inventors have recently demonstrated the effectiveness of fasting or FMD cycles in combination with chemotherapy to reduce tumor growth in a wide range of cancer types compared to standard chemotherapy alone (Lee et al., 2012; Di Biase et al., 2016).

Since KRAS mutant cancer are often resistant to conventional treatments, the inventors investigated whether FMD and vitamin C combination (or combo) therapy, possibly by increasing cellular oxidative stress, could sensitize KRAS mutant cancer cells to the pro-oxidant action of chemotherapy in vivo. Mice bearing HCT116 xenografts were fed ad libitum with standard rodent diet or subjected to FMD cycles (3-days each) and treated with high dose vitamin C (4 g/kg i.p.) or saline, oxaliplatin (10 mg/kg) or vehicle, as single agents or in combination. Interestingly, the inventors found that FMD and vitamin C combo treatment is as effective as oxaliplatin combined with FMD (FMD+vitamin C vs FMD+oxaliplatin) or vitamin C (FMD+vitamin C vs vitamin C+oxaliplatin), thus supporting the powerful action of these non-toxic combination in reducing tumor growth (FIG. 17). Importantly, combination of FMD cycles, vitamin C and chemotherapy as triple treatment was the most effective therapeutic option in delaying tumor progression (FIG. 17).

Fasting or Short-Term Starvation (STS) reduces tumor progression and sensitizes different types of cancers to chemotherapy, while protecting normal cells through a mechanism, which partially involves IGF-1 reduction (Lee et al., 2012). These phenomena are described as “Differential Stress Sensitization” (DSS) and “Differential Stress Resistance” (DSR), respectively (Raffaghello et al., 2008; Lee et al., 2010; Lee and Longo, 2011; Lee et al., 2012; Longo and Mattson, 2014). In addition, oxidative stress results to contribute to fasting effects, in fact, by increasing ROS generation, fasting sensitizes cancer cells to the oxidative damages exerted by chemotherapy (Lee et al., 2012).

However, fasting remains a challenging option for cancer patients, thus encouraging the clinical use of a fasting-mimicking diet (FMD), which represents a more feasible therapeutic intervention (Brandhorst et al., 2015; Di Biase et al., 2016).

The inventors discovered a very low toxicity combination therapy for the treatment of the highly aggressive KRAS mutant cancers.

KRAS mutant tumors are refractory to standard and targeted treatment, making the patient's prognosis very poor (Lievre et al., 2006). For this reason, there is an increasing and urgent need to identify effective therapeutic option able to delay or eradicate KRAS-driven tumor progression. However, several attempts have failed in efficacy and specificity for the treatment of this type of cancers so far (Cox et al., 2014; Verissimo et al., 2016). Indeed, patients bearing KRAS-mutant CRC result to be unresponsive to targeted-therapy, such as monoclonal antibody directed against EGFR (cetuximab and panitumumab), and still now, no effective therapeutic options are available. In fact, despite several improvements have been achieved, the survival rate at 5 years post diagnosis remains very poor (Bardelli and Siena, 2010; Brenner et al., 2014).

Recently, Yun and colleagues reported that high-dose vitamin C is effective in killing KRAS mutant CRC cells, arising the possibility that vitamin C could have a potential therapeutic use for the treatment of this aggressive tumor type (Yun et al., 2015).

The anti-cancer properties of high-dose vitamin C have been associated with controversial results. In 1976, vitamin C was proposed by Cameron and Pauling as an anti-tumoral agent, however two randomized clinical trials failed to demonstrate any beneficial effect of oral-administered vitamin C on cancer patient survival (Cameron and Pauling, 1976; Creagan et al., 1979; Moertel et al., 1985). These contradictory outcomes are explained, at least in part, by the different administration route. In fact, growing evidence sustains that vitamin C requires to be delivered intravenously in order to bypass the gastric barrier and achieve plasma millimolar concentrations, which are toxic to cancer cells (Padayatty et al., 2010; Chen et al., 2008, Stephenson et al., 2013). For this reason, several studies are now focusing on vitamin C mechanism of action and on its effectiveness as anti-cancer drug, through several pre-clinical and clinical trials (Hoffer et al., 2008; Ma et al., 2014; Schoenfeld et al., 2017, NCT02344355; NCT03146962; NCT02420314; NCT01752491). Present data support previous published findings showing that KRAS mutant cancer cells are more susceptible to vitamin C toxicity, compared to KRAS wild type cell lines. However, vitamin C anti-cancer effect is limited, only slightly increasing cell death in vitro and retarding tumor progression in vivo (Hoffer et al., 2008; Padayatty et al., 2010; Stephenson et al., 2013; Welsh et al., 2013; Ma et al., 2014). For this reason, present goal was to evaluate whether vitamin C toxicity could be potentiated by FMD cycles.

Importantly, the inventors discovered that in vitro STS is able to act synergistically with vitamin C in causing toxicity to a wide range of KRAS mutant cell lines derived from colorectal, lung and pancreatic cancers, leaving unaffected KRAS wild type prostate and ovary cancer cells, normal colon cells and normal fibroblasts. These findings suggest that KRAS oncogenic reprogramming is required for the FMD-mediated sensitization to vitamin C toxicity. At molecular level, the inventors discovered that the observed effect is due to, at least partially, a dramatic increase in ROS and free iron levels upon STS and vitamin C co-treatment.

In mouse models, FMD cycles combined with pharmacological vitamin C treatment are more effective than vitamin C and FMD as single interventions in delaying tumor progression. Moreover, given the tolerability and efficacy of FMD and vitamin C as anti-cancer agents, the inventors aim at translating these pre-clinical studies into the clinic, as potential novel therapeutic option for KRAS mutant cancer patients.

Given the promising results on the safety and effectiveness of pharmacological vitamin C integration in standard therapy, the inventors evaluated whether a more potent intervention as FMD and vitamin C combo treatment, could potentiate chemotherapy efficacy in KRAS mutant cancer patients that are unresponsive to conventional therapeutic agents.

The standard care for KRAS mutant CRC patients often includes oxaliplatin, a DNA synthesis inhibitor, with limited efficacy. Importantly, the inventors discovered that FMD and vitamin C combinatorial treatment potentiates chemotherapy effectiveness in delaying tumor progression in mouse model.

In conclusion, the inventors discovered that FMD and vitamin C combination treatment represents a safe therapeutic option which can be easily integrated with standard therapy, to ameliorate the prognosis for patients bearing KRAS-driven cancers. Furthermore, present pre-clinical results support the use of the combination of FMD, vitamin C and oxaliplatin triple treatment on KRAS mutant CRC patients.

Materials and Methods

Cell Lines and Culture Conditions

HCT116, HT29, NCI-H23 and PC-3 cells were obtained from NCI 60 panel; CT26.WT and CCD-84CoN cells were purchased from ATCC; DLD1 cell line was purchased from DSMZ; NCI-H727, PANC-1 and Cov362 cells were purchased from ECACC. All cell lines were maintained in Dulbecco's Modified Eagle Medium (DMEM) (Life Technologies, Cat. #: 10566) supplemented with 10% FBS (Biowest, Cat. #: S1810), 1% non-essential amino acids (Biowest, Cat. #: X-0557), and 1% penicillin/streptomycin (Biowest, Cat. #L0022). All cells were tested for mycoplasma contamination routinely. Cells were maintained in a humidified, 5% CO₂ atmosphere at 37° C.

For FMD-like condition experiments, cells were grown in DMEM medium without glucose (DMEM no glucose, Life Technologies, Cat. #: 11966025) supplemented with 0.5 g/l glucose (Sigma-Aldrich, Cat. #: G8769) and 1% FBS, referred as Short-Term starvation medium (STS). For mimicking standard condition, cells were grown in DMEM medium without glucose (DMEM no glucose, Life Technologies, Cat. #: 11966025) supplemented with 1 g/l glucose (Sigma-Aldrich, Cat. #: G8769) and 10% FBS, referred as control medium (CTR).

Reagent Preparations

Oxaliplatin

Oxaliplatin was kindly provided by the IEO hospital pharmacy (Milan). The stock solution (5 mg/ml) was dissolved in solution for injections (water, tartaric acid, sodium hydroxide).

Vitamin C

Sodium ascorbate was purchased from Sigma-Aldrich (Cat. #: A4034) and was dissolved in sterile saline. Stock solutions of 20 mg/ml or 240 mg/ml were prepared for in vitro and in vivo experiments, respectively. Vitamin C was freshly prepared each time before use.

Glutathione

Reduced glutathione (GSH) was purchased from Sigma-Aldrich (Cat. #: G6013) and dissolved in sterile water to a final concentration of 32.5 mM (stock solution). Stock solutions were stored at −20° C.

N-Acetyl Cysteine

N-acetyl cysteine (NAC) was purchased from Sigma-Aldrich (Cat. #: A9165) and dissolved in sterile water to a final concentration of 100 mM (stock solution). Stock solutions were freshly prepared for each experiment.

Desferrioxamine

Desferrioxamine (DFO) was purchased from Sigma-Aldrich (Cat. #: D9533) and dissolved in sterile deionized water to final concentration of 40 mg/ml. Stock solutions were stored at −20° C.

Hemin Hemin was purchased from Sigma-Aldrich (Cat. #: 51280) and dissolved in 1.4 M NH₄OH (Sigma-Aldrich, Cat. #: 221228) to final concentration of 25 mg/ml (stock solution). Stock solutions were stored at +4° C.

Zinc Protoporphyrin

Zinc protoporphyrin (ZnPP) was purchased from Sigma-Aldrich (Cat. #: 282820) and dissolved in DMSO to final concentration of 25 mg/ml. Stock solutions were stored at −20° C.

Catalase

Catalase (Cat) from bovine liver (2000-5000 U/ml) was purchased from Sigma-Aldrich (Cat. #: C1345) and dissolved in 50 mM potassium phosphate buffer to a final concentration of 5000 U/ml. Stock solutions were freshly prepared for each experiment.

MnTMPyP

MnTMPyP (superoxide dismutase mimetic) was purchased from Merck Millipore (Cat. #: 475872) and dissolved in sterile deionized water to a final concentration of 0.697 mM. Stock solutions were stored at −20° C.

Viability Assays

MTT Assay

The MTT [3-(4,5-Dimethylthiazol-2-yl)-2,5-diphenyltetrazolium bromide] assay is a quantitative colorimetric assay used to determine cell viability (Mosmann, 1983). MTT is characterized by a yellow colour, and upon its reduction to formazan by mitochondrial dehydrogenases, turns into a purple molecule, which has an absorbance peak at 570 nm. The absorbance is proportional to the amount of formazan produced, thus giving a measure of living cells, since the reaction only occur in living and metabolically active cells (Mosmann, 1983).

To evaluate viability, cells were seeded into a 96-well plate (2000 cells per well) in CTR medium. 24 hours later, cells were rinsed twice in 1×PBS, and CTR or STS medium was added. After 24 hours, media were refreshed to ensure that glucose and serum levels were not completely exhausted, and cells were treated with 40 μM oxaliplatin or vehicle for further 24 hours. At the end of the experiment, medium was removed and cells were incubated with 100 μl of MTT (Sigma-Aldrich, Cat. #: M2128) solution (0.5 mg/ml, dissolved in medium) per well, for 3 hours at 37° C. in the dark. Next, 100 μl of lysis buffer (10% SDS, HCl 0.01M) were added to each well to dissolve formazan crystals and plates were incubated overnight at 37° C. Absorbance was recorded at the wavelength of 570 nm using a microplate reader (Infinite M200 TECAN). The absorbance values of media background were subtracted from the absorbance of each sample. No-treated samples were used as reference values (100% survival) to normalize the absorbance of treated samples.

Erythrosin B Exclusion Assay

Erythrosin B is a vital dye that is impermeable to biological membranes, thus staining only unviable cells with disintegrated membranes (Kim et al., 2016).

To measure cell death, cells were seeded in 12-well plates at a concentration of 34000 cells per well in CTR medium. The next day, cells were rinsed twice in 1×PBS and CTR or STS medium was added. After 24 hours, media were refreshed to ensure that glucose and serum levels were not completely exhausted and cells were treated with 40 μM oxaliplatin or vehicle for further 24 hours. At the end of the experiment, cells were harvested by trypsinization, centrifuged and resuspended in the respective media for a final concentration of 1×10⁶ cells per ml. Cell death was measured by erythrosine B exclusion assay. Cell suspension was diluted 1:1 with erythrosin B 0.1% in PBS (Sigma-Aldrich, Cat. #: 200964), then cells were counted in a Bürker chamber, and percentage of cell death was calculated as the number of Erythrosin B-positive cells with respect to the total number of cells.

Muse Cell Analyzer

The Muse cell analyser is a fluorescent-based microcapillary cytometer for single cell analysis (Merck Millipore). The Muse viability assay kit (Merck Millipore, Cat. #: MCH100102) uses a single reagent containing 2 fluorescent dyes, which intercalates DNA molecules. One dye is cell permeable, and stains all nucleated cells, allowing to discriminate cells from debris; whereas the second dye stains only cells with compromised membrane, giving a measurement of cells that are dead or are dying.

For cell death measurement, cells were seeded in 12-well plates at a concentration between 20′000 to 80′000 cells according to the cell line, so that at the moment of vitamin C treatment, cells reach 40% of confluence. 24 hours after seeding, cells were rinsed twice in PBS and then grown in CTR or STS medium. After 24 hours, media were refreshed to ensure that glucose and serum levels were not completely exhausted, and after medium pH stabilization at 37° C. and 5% CO₂ atmosphere, cells were treated with 350 μM vitamin C or vehicle (deionized water) for the next 24 hours. For experiments with anti-oxidant agents, cells were treated with 5 mM glutathione, and 5 mM N-acetyl cysteine, together with vitamin C. For experiments with desferrioxamine, 500 μM desferrioxamine was added 6 hours before vitamin C treatment for HCT116 cells or 12 hours for DLD1 and CT26.WT. After the specific incubation time, cells were washed twice in PBS to evaluate only the intracellular effect of DFO, and then, CTR or STS fresh medium and vitamin C were added for the next 24 hours for HCT116 and DLD1 cells, and 9 hours for CT26.WT. For hydrogen peroxide scavenging experiments, cells were treated with catalase from bovine liver (50 U/ml) 1 minute before vitamin C was provided as previously described (Schoenfeld et al., 2017) or with MnTMPyP, 2 hours before vitamin C administration. For HO-1 activation experiments, cells were treated with hemin at a concentration of 20 μM, 3 hours before vitamin C was provided. For HO-1 inhibition experiments, cells were treated with zinc protoporphyrin (ZnPP) at a concentration of 20 μM, 3 hours before vitamin C At the end of the experiment cells were harvested by trypsinization, centrifuged and resuspended in the respective media for a final concentration of 1×10⁶ cells per ml. Cell suspension and Muse viability reagent are mixed in 1:10 ratio and after 5 minutes of incubation in the dark, viability was analysed by Muse cell analyser. Data are expressed as percentage of dead cells.

Reactive Oxygen Species Measurement

Intracellular reactive-oxygen species (ROS) were measured using the CellROX oxidative stress reagents (Life Technologies, Cat. #: 10422).

CellROX fluorogenic probe is designed to measure reactive-oxygen species (ROS) in live cells. The probe is cell permeable and in reduced state it is no or weakly fluorescent, whereas upon oxidation it shows a fluorogenic signal. CellROX probe exhibits a fluorescence excitation at 640 nm and fluorescent emission at 665 nm (deep red).

For ROS measurement, cells were seeded in 10 cm petri dish (4×10⁵ cells in CTR medium). After 24 hours cells were rinsed twice in 1×PBS and CTR or STS medium was added. 24 hours later, cells were trypsinized, resuspended in their respective media, and treated with 1 mM sodium ascorbate and 1 μM CellROX deep red reagent in the dark, for 30 minutes at 37° C. and 5% CO₂. Then, fluorescence was immediately analyzed by flow cytometry (Attune NxT flow cytometer). Data were processed by Kaluza analysis software (Beckman coulter, version 2.0). Data were expressed as fold change of the median fluorescent intensity (MFI) of each treated sample versus the MFI of control sample.

Intracellular Ferrous Iron Detection

Intracellular ferrous ions were measured using Iron Assay Kit (Cat. #: ab83366). Ferrous ions (Fe²⁺), but not ferric ions (Fe³⁺) specifically reacts with ferene-S (an iron chromogen) producing a stable colored complex, whose absorbance is measured at 593 nm.

For Fe²⁺ measurement experiment, cells were seeded in 10 cm petri dish (4×10⁵ cells) in CTR medium. After 24 hours cells were rinsed twice in 1×PBS and CTR or STS medium was added. 24 hours later, medium was refreshed and cells were treated with 350 μM vitamin C or vehicle for 3 hours. Next, cells were lysed in iron assay buffer and intracellular ferrous iron level was measured according to manufacturer protocol. Absorbance was recorded at the wavelength of 593 nm using a microplate reader (Infinite M200 TECAN). Data are expressed as fold change versus control sample.

RNA Interference with siRNA Oligonucleotides

RNA interference was carried out on HCT116 using Lipofectamine RNAiMAX (Invitrogen, Cat. #: 13778150) following supplier's protocol. HCT116 cells were seeded the day before transfection and transfected at 50-60% confluence with the indicated siRNA oligonucleotides (25 nM). The following oligonucleotides were used: ON-TARGETplus human HO-1 siRNA (pool of four siRNA) and ON-TARGETplus non-targeting pool (as a negative control) (Dharmacon). Knockdown efficiency was assessed by western blot analysis.

Protein Extraction and Western Blot Analysis

Cells were washed twice in ice-cold PBS and lysates were prepared in RIPA lysis buffer (50 mM Tris HCl pH 7.4, 150 mM NaCl, 1% NP-40, 0.25% deoxycholic acid, 1 mM EDTA) supplemented with protease and phosphatase inhibitors (protease inhibitor cocktail set III EDTA-free, Calbiochem, Cat. #: S39134; PhosStop, Roche), Samples were sonicated (bioruptor plus, diogenode) and centrifuged at 16.1 g for 30 minutes at 4° C. Tumor tissues were collected and snap frozen in liquid nitrogen immediately after mice were sacrificed, and stored in −80° C. until use. For protein extraction, tumors were homogenized with Tissue lyser II (Qiagen) in RIPA buffer supplemented with protease and phosphatase inhibitors and then ultra-centrifuged (45000 rpm using a MLA-130 Beckman rotor) for 1 hour. Protein concentrations were determined by BCA assay (Thermo Fisher Scientific, Cat. #: 23225). Proteins were diluted in 1× Laemmli sample buffer, boiled at 95° C. for 5 minutes and 30 μg of total proteins were separated by using SDS-PAGE and analyzed by western blotting by standard procedures. After protein transfer, the nitrocellulose membranes with 0.22 μm pore size (GE Healthcare Life Sciences, Fisher Scientific) were blocked by incubation with 5% non-fat dry milk in 1×0 TBS-Tween (0.1%) for 1 hour at room temperature. Membranes were incubated overnight at 4° C. with gentle shaking with the following primary antibodies: HO-1 (1:1000, Enzo Life Science, Cat. #: ADI-SPA894), FTH (1:1000, Cell Signaling, Cat. #: 3998), AMPK (1:1000, Cell Signaling, Cat. #: 2532), phospho-threonine 172 AMPK (1:1000, Cell Signaling, Cat. #: 2535), eIF2a (1:1000, Cell signaling, Cat. #: 5324), phospho serine 51 eIF2a (1:1000, Cell signaling, Cat. #: 3597), AKT (1:1000, Cell Signaling, Cat. #: 9272), phospho-serine 473 AKT (1:1000, Cell Signaling, Cat. #: 9271), H2AX (1:4000, Abcam, Cat. #: ab11175), phospho serine 139 H2AX (1:5000, Merck Millipore, Cat. #: 05636), Vinculin (1:10000, Sigma-Aldrich, Cat. #: V9131), (3-actin (1:3000, Sigma, Cat. #: A2066).

Next, membranes were washed 3 times (5 minutes each) with TBS-Tween (0.1%) and then incubated for 1 hour at room temperature with the following secondary antibodies linked to horseradish peroxidase (Biorad): anti-Mouse (1:3000, Cat. #: 170-6516) and anti-Rabbit (1:3000, Cat. #: 170-6515). After 3 washes in TBS-Tween, immunostained bands were detected under a ChemiDoc imaging system (Biorad) using the chemiluminescent method (super signal west PICO and super signal west DURA, Thermo Fisher, Cat. #: 34577, Cat. #: 34075). Bands intensity was quantified with NIH Image J software (version 1.48v).

RNA Extraction, RT-PCR and qRT-PCR

Total RNA was isolated using the miRNeasy Mini Kit (QIAGEN, #217004) according to the manufacturer's instructions. Next, 1 μg of purified RNA was retro-transcribed by using SuperScript Vilo cDNA synthesis kit (Invitrogen, #11754050).

Resulting cDNA (1/20 v/v) was analyzed by real-time polymerase reaction (qRT-PCR) using TaqMan MBG probes with FAM reporter dyes. Human target gene primers for heme-oxygenase-1 (HMOX1:Hs01110250_ml, ThermoFisher Scientific) were utilized. Target transcript levels were normalized to those of a reference gene (GAPDH: hs99999905_m1, ThermoFisher Scientific).

In Vivo Procedures

Mouse Models

The animals were housed under specific pathogen-free conditions with 12 hours day/light cycles. All experiments were performed in accordance with the guidelines established in the Principles of Laboratory Animal Care (directive 86/609/EEC) and were approved by the Italian Ministry of Health.

For xenograft experiments, 8-weeks old female NOD scid gamma (NSG, Charles River) were subcutaneously injected with 2×10⁶ HCT116 cells (NCI 60 panel) resuspended in 100 μl of PBS. For syngeneic model, 8-weeks old female Balbc/Ola Hsd mice (Envigo) were subcutaneously injected with 3×10⁵ CT26 wt (ATCC) cells resuspended in 100 μl of PBS. When tumors were palpable (approximately 7 days after inoculation), mice were randomly divided in the different experimental groups. Body weights were recorded daily, and tumor volumes were measured every 2-3 days by a digital vernier caliper according to the following equation: tumor volume (mm³)=(length×width²)×0.5, where the length and width are expressed in millimetres. At the end of the experiments, mice were euthanized by using CO₂.

Animal Diets and Treatments

Mice were fed ad libitum with irradiated VRFI (P) diet (Charles River) containing 3.89 kcal/g of gross energy.

Presntfasting-mimicking diet (FMD) diet is based on a nutritional screen that identified ingredients that allow nourishment during periods of low calorie consumption (Brandhorst et al., 2015). The FMD diet consists of two different components designated as day 1 diet and days 2-4 diet. Day 1 diet contains 7.67 kJ/g (provided 50% of normal daily intake; 0.46 kJ/g protein, 2.2 kJ/g carbohydrate, 5.00 kJ/g fat); the day 2-4 diet contains 1.48 kJ/g (provided at 10% of normal daily intake; 0.01 kJ/g protein/fat, 1.47 kJ/g carbohydrates). Before FMD diet was supplied, mice were transferred in fresh cages to avoid residual chow feeding and coprophagy. Mouse weight was monitored daily and during FMD cycle weight loss did not exceed 20%.

For experiments on tumor growth, mice were fed with standard rodent diet or underwent FMD cycles. Each FMD cycle lasts 3 days because of the faster rate of body weight loss of these mouse strains compared to others. In experiments with chemotherapy, the second FMD cycle was reduced to 2 days because of chemotherapy-induced body weight loss. For FMD cycles lasting 2 or 3 days, day 1 diet contains 7.67 kJ/g (provided 50% of normal daily intake; 0.46 kJ/g protein, 2.2 kJ/g carbohydrate, 5.00 kJ/g fat); day 2 (and day 3 where present) contains 1.48 kJ/g (provided at 10% of normal daily intake; 0.01 kJ/g protein/fat, 1.47 kJ/g carbohydrates). Before FMD cycle was repeated, mice completely recovered their original bodyweight.

For vitamin C experiments, mice undergoing standard feeding or at the last day of the first FMD cycle started to be treated with vitamin C (4 g/kg in saline) via intraperitoneal injection twice a day, every day until the end of the experiment. At least 6-8 hours have elapsed between the two administrations in each day.

For chemotherapy experiments, mice undergoing standard feeding or at the second day of the first FMD cycle started to be treated with vitamin C twice a day every day until the end of the experiment, and during the last day of each FMD cycle (24 hours before refeeding), mice were treated with oxaliplatin (10 mg/kg) via intraperitoneal injection. When mice were treated with chemotherapy, vitamin C injection was skipped. At least 8-9 hours have elapsed between oxaliplatin and vitamin C administrations.

Statistical Analysis

GraphPad Prism v6.0c was used for the analysis of the data and graphic representations. Comparisons between groups were performed with two-sided unpaired Student's t-test and p values≤0.05 were considered significant.

One-way ANOVA analysis was used for comparison among multiple groups for mouse experiments. One-way ANOVA analysis was followed by Tukey's test post analysis. P values≤0.05 were considered significant.

For clinical data analysis, Wilcoxon matched-pairs signed rank test was utilized.

For the quantification of signals (western blot bands), NIH Image J software (version 1.48v) was utilized.

All data are represented as mean±SEM of at least three independent experiments.

### Example 2

FMD or Fasting Cycles Delay Tumor Progression and Enhance Vitamin C Anti-Cancer Effect in KRAS Mutant CRC Mouse Models

Supported by their in vitro results, inventors investigated whether fasting or FMD cycles could enhance vitamin C toxicity also in mouse models of KRAS mutant colorectal cancer. FMD is a low calories, protein and sugar but high unsaturated fat diet, which is able, as fasting, to delay tumor progression and sensitize cancer cells to chemotherapy (Brandhorst et al., 2015; Di Biase et al., 2016).

First, they tested the effect of complete fasting in combination with high dose vitamin C on human KRAS-driven colorectal cancer cells in vivo. For this purpose, nod scid mice (NSG) bearing HCT116 subcutaneous tumors, were fed ad libitum with standard rodent diet or fasted (water only) for two days every week.

It has been already described that the parenteral administration mediated by intraperitoneal injection, allows to reach high-dose vitamin C (3 mM), which is necessary to obtain the anti-cancer effect (Chen et al., 2007; Chen et al., 2008). Thus, mice were treated with saline or high dose vitamin C (4 g/kg) intraperitoneally (i.p.). twice a day, every day, as previously described (Chen et al., 2007; Chen et al., 2008).

Present results show that three cycles of fasting were as effective as pharmacological vitamin C in reducing tumor progression, however when fasting cycles were combined with vitamin C the anti-cancer effect was significantly enhanced (FIG. 18).

However, fasting remains a challenging option for cancer patients, thus encouraging the pre-clinical and clinical use of FMD, which represents a more feasible therapeutic intervention (Brandhorst et al., 2015; Wei et al., 2017).

FMD-Mediated Sensitization to Pharmacological Vitamin C Acts Through ROS Generation

STS and Vitamin C Combo Treatment Induces Marker of Metabolic Stress

STS sensitizes CRC cells to chemotherapy-associated oxidative damage in part by exerting an “anti-Warburg” effect (Bianchi et al., 2015). Glycolysis and glutaminolysis reduction, together with the enhancement of OXPHOS uncoupled from energy production, lead to the inhibition of ATP synthesis and an increase in ROS levels (Bianchi et al., 2015).

To explore whether energetic impairment and oxidative stress could be involved in STS-mediated cell death induction upon vitamin C treatment, inventors evaluated the phosphorylation level of the AMP-activated protein kinase (AMPK). AMPK represents a sensor of energy levels, which is activated by phosphorylation upon reduction of ATP/AMP ratio (Hardie et al., 2012). Interestingly, inventors found that STS in combination with vitamin C, strongly up-regulated AMPK phosphorylation level, indicating a possible presence of energetic stress (FIG. 19). Importantly, in addition to adenylate pool alteration, AMPK results to be activated in response to pro-oxidant conditions, such as increases of ROS levels (Cardaci et al., 2012). It has been shown that sustained AMPK activation induces cell death through, at least in part, the downregulation of AKT activity (Tzatsos and Tsichlis, 2007).

AKT is a serine-threonine kinase involved in the control of different biological processes, including metabolism, proliferation and survival, in response to several growth factors (Manning and Cantley, 2007). Importantly, the inventors have previously shown that STS exerts its effects by affecting pro-growing signalling pathways, such as PI3K/AKT pathway, which in turn contribute to metabolic reprogramming (Bianchi et al., 2015).

AKT activity, measured by its phosphorylation level, was downregulated upon STS. More importantly, when combined with vitamin C, this effect was potentiated even further (FIG. 20).

In agreement with a reduction of the pro-growth PI3K/AKT pathway, STS and vitamin C combo treatment significantly induced the phosphorylation level of the eukaryotic initiation factor 2 alpha (eIF2a) (FIG. 21), which is a marker of global protein synthesis inhibition (Kimball, 1999).

Taken together, these results indicate that the combinatorial treatment causes a dramatic cell death induction in part by inhibiting crucial processes for viability.

Oxidative Stress is the Causative Factor for FMD and Vitamin C Combo Treatment Toxicity in KRAS Mutant CRC Cells

The metabolic stress induction, indicated by the alteration of AMPK and AKT levels, prompted the inventors to investigate whether ROS generation could be involved. ROS, which include H₂O₂ and superoxide (O₂.⁻), are generated as by-products of normal metabolisms. However, excessive ROS production, known as oxidative stress, have detrimental effects on cellular biomolecules, including DNA, lipids and proteins (Reczek and Chandel, 2017).

The observation that STS and vitamin C combo treatment strongly induced DNA damage, measured by the phosphorylation level of the histone H2AX, further support the hypothesis that oxidative stress participates in mediating the cytotoxic effect (FIG. 22).

To explore this hypothesis, ROS levels were measured as indicated by the oxidation of the fluorogenic probe CellROX, in HCT116 cells grown in CTR or STS condition. As show in FIG. 23, STS was able to increase ROS level by nearly 2-fold compared to CTR condition (FIG. 23). However, inventors did not observe induction of DNA damage marker phospho-H2AX or cell death in STS condition as single intervention, thus indicating that the increased ROS level are handled by KRAS mutant cells (FIG. 22). In order to respond to oxidative stress and prevent oxidative damages normal and cancer cells express a large array of anti-oxidant enzymes (Kimmelman, 2015).

The inventors performed a SILAC (stable isotope labeling with amino acids in cell culture) coupled to LC-MS/MS (Liquid chromatography-mass spectrometry/mass spectrometry) analysis on HCT116 cells grown in CTR or STS condition (FIG. 32). Briefly, HCT116 cells that have incorporated light or heavy isotope labelled arginine and lysine, were cultured in CTR and STS medium. After labelling, light and heavy isotope labelled proteins are mixed and analysed by LC-MS/MS. The relative abundance of proteins in each condition was measured from the relative intensity of the light and heavy peptides.

The inventors checked the expression of different antioxidant enzymes in HCT116 cells grown in an FMD-like condition and the inventors observed an increase in the expression of GSTO1, PRDX2 PRDXS, APOM, MGST1, NQO1 and SOD1 (Table 1), which may suggest a response to the increase in ROS levels. Gene ontology analysis on proteins up-regulated in STS condition reveals also a significative enrichment in molecular processes involved in anti-oxidant response (oxidoreductase activity, acting on CH—CH group of donors [GO:0016628], p=0.001496; oxidoreductase activity, acting on CH—OH group of donors [GO:0016616], p=0.005658; oxidoreductase activity, acting on the aldehyde or oxo group of donors [GO:0016620], p=0.03018).

These findings, together with the extensively demonstrated role of vitamin C pro-oxidant action (Chen et al., 2008; Yun et al., 2015), raise the possibility that a high level of oxidative stress may be involved in FMD-mediated sensitization to vitamin C.

To explore this hypothesis, ROS levels were measured in HCT116 cells grown in CTR or STS condition, in presence or absence of vitamin C Notably, my results indicate that STS as single intervention was able to increase ROS levels, as well as vitamin C However, the combinatorial treatment strongly exacerbated cellular oxidative state by 3.6-fold (FIG. 23).

To directly test whether high ROS level is a causative or a secondary event in STS-induced sensitization to vitamin C, KRAS mutant CRC cells (HCT116, DLD1, CT26.WT) grown in STS condition and exposed to vitamin C, were co-treated with glutathione (GSH), which is the major cellular antioxidant, as well as with the cell-permeable reducing agent and glutathione precursor N-acetyl cysteine (NAC). Confirming previous findings (Yun et al., 2015), both agents rescued vitamin C cytotoxic effect in CTR medium. Interestingly, GSH and NAC were also able to revert the massive cell death induction mediated by STS and vitamin C cotreatment, suggesting that STS may act by exacerbating the pro-oxidant action of vitamin C (FIG. 24). Accordingly, GSH cotreatment fully reversed the increase in ROS mediated by STS and STS+vitamin C in HCT116 cell line (FIG. 25). These findings support the hypothesis that ROS production could represent the main route through which STS acts.

Next, to further assess whether oxidative stress is the causative event in mediating STS+vitamin C cytotoxicity, inventors tested the effect of exogenous membrane-impermeable catalase, a well-known ROS scavenger able to specifically decompose hydrogen peroxide to water and molecular oxygen (Chelikani et al., 2004). Present data show that exogenous catalase exposure prior vitamin C treatment, was able to revert STS-mediated sensitization to vitamin C (FIG. 26A). In addition, the treatment with the membrane-permeable superoxide dismutase (SOD)/catalase mimetic MnTMPyP, which has been reported to scavenge both superoxide anions and also hydrogen peroxide, before vitamin C exposure, inhibited cell death upon combinatorial treatment (FIG. 26B).

Collectively present data strongly indicate that increased ROS levels are the causative factors responsible for STS-mediated sensitization to vitamin C anti-cancer effect.

Importantly, the results obtained in vitro were also confirmed in vivo, where inventors found that FMD cycles down-regulated FTH protein expression level also in HCT116-derived grafted tumors (FIG. 9).

Heme-Oxygenase-1 Down-Regulation is Central in FMD-Mediated Sensitization to High Dose Vitamin C

Vitamin C Up-Regulates HO-1 Expression Level while FMD Reverts this Effect

Several studies have described the role of ferritin in protecting cells from oxidative damages by sequestering LIP (Torti and Torti, 2002).

Among the protective enzymes, which are involved in response to oxidative insults, the stress-inducible protein heme oxygenase-1 (HO-1) exerts its anti-apoptotic function in part by inducing ferritin expression (Ferris et al., 1999; Gonzales et al., 2002).

Supported by present data showing that FMD down-regulates FTH protein expression level, inventors investigated whether HO-1 could be involved in altering cellular iron content during FMD and vitamin C combo treatment. Inventors evaluated the association between HO-1 expression level and FTH induction in their model system. To this purpose, HCT116 cells were treated with the HO-1-activator hemin, confirming that hemin increases both HO-1 and FTH protein expression level in CTR and STS growing conditions (FIG. 27).

FMD, Vitamin C, Oxaliplatin Triple Treatment is Effective in Reducing Progression of KRAS Mutant CRC

In Vitro STS Condition Sensitizes KRAS Mutant CRC Cells to Chemotherapy

The inventors have recently shown that fasting and FMD are effective non-toxic interventions able to sensitize a wide range of cancer cells to the cytotoxic effect of chemotherapy, while protecting normal cells, through a mechanism which partially involve the lowering of IGF-1 level (Lee et al., 2012; Brandhorst et al., 2015, Di Biase et al., 2016).

To evaluate whether FMD potentiates chemotherapy efficacy in thier in vitro model system, inventors used the chemotherapeutic agent oxaliplatin, which represents a first-line standard drug for oncological patient bearing KRAS mutant CRC (Brenner et al., 2014). Present in vitro data on HCT116 cell line indicate that 24 hours-STS before and during oxaliplatin treatment is able to significantly decrease the percentage of metabolically active cells, as measured by MTT assay, and increase the percentage of cell death (FIG. 30). Notably, the combination of a FMD-like condition and chemotherapy was also more effective compared to 48 hours-STS as single intervention or chemotherapy alone in decreasing viability of HCT116 cells, thus encouraging the use of FMD as an effective combination intervention in KRAS mutant cancers. (FIG. 28).

FMD and Vitamin C Combo Treatment Potentiates Oxaliplatin Efficacy in Mouse Models of KRAS Mutant CRC

Next, the anti-cancer effect of the triple therapy was also evaluated in a syngeneic mouse model of KRAS mutant CRC (CT26.WT allograft). A first preliminary experiment showed that FMD and vitamin C combo treatment resulted to be as effective as oxaliplatin and vitamin C dual therapy in retarding tumor growth. Importantly the triple treatment (FMD+vitamin C+oxaliplatin) further delayed tumor progression, thus indicating its efficacy also in an immuno-competent model of KRAS mutant CRC (FIG. 29).

Finally, the combination of FMD cycles, vitamin C and oxaliplatin results to be well tolerated, as indicated by mouse bodyweight. FMD cycles causes a 20% weight loss, which is recovered upon refeeding, independently from vitamin C and oxaliplatin administration (FIG. 30).

Fasting or Short-Term Starvation (STS) reduces tumor progression and sensitizes different types of cancers to chemotherapy, while protecting normal cells (Lee et al., 2012; Bianchi et al., 2015). These phenomena are described as “Differential Stress Sensitization” (DSS) and “Differential Stress Resistance” (DSR), respectively (Raffaghello et al., 2008; Lee et al., 2010; Lee and Longo, 2011; Lee et al., 2012; Longo and Mattson, 2014). The differential protection of normal and cancer cells in response to fasting has been proposed to be mediated, at least in part, by the down-regulation of the insulin-like growth factor (IGF-1) pathway (Lee et al., 2010). By decreasing IGF-1 level, fasting allows normal cells to enter in a protection mode, reducing mitotic stimuli and inducing the expression of repair genes (Longo and Mattson, 2014; Raffaghello et al., 2008). Cancer cells, instead, harbouring oncogenic mutations, are unresponsive to growth signals, and thus fail to be protected (Lee et al., 2012; Lee et al., 2010). Studies showing that IGF-1 restoration reverts the fasting-mediated sensitization to chemotoxicity, sustain its fundamental role also in DSS (Lee et al., 2010; Lee et al., 2012). In addition, oxidative stress results to contribute to fasting effects, in fact, by increasing ROS generation, fasting sensitizes cancer cells to the oxidative damages exerted by chemotherapy (Lee et al., 2012).

However, fasting remains a challenging option for cancer patients, for this reason inventors developed a more feasible and safer diet whose specific formulation mimics fasting effects. The fasting-mimicking diet (FMD) is a low calories, protein and sugar but high unsaturated fat diet with effects similar to fasting in delaying tumor progression and in sensitizing cancer cells to chemotherapy, as shown in breast cancer and melanoma models (Brandhorst et al., 2015; Di Biase et al., 2016).

The present results focused on the identification of very low toxicity combination therapy, with the potential to 1) replace chemotherapy and other high toxicity therapies, 2) lead to cancer free survival or high therapeutic index in combination with toxic therapies. For this purpose, inventors focused on the therapeutic effect of FMD on the highly aggressive KRAS mutant colorectal cancer, which is one of the most widespread and lethal cancer in the western world (Bardelli and Siena, 2010; Stephen et al., 2014; Van Emburgh et al., 2016).

KRAS mutant cancers are refractory to standard and targeted treatment, making the patient's prognosis very poor (Lievre et al., 2006). For this reason, there is an increasing and urgent need to identify effective therapeutic option able to delay or stop KRAS-driven tumor progression without causing the major side effects that eventually lead to death or force oncologist to discontinue treatment. However, several attempts have failed in efficacy and specificity for the treatment of this type of cancer so far (Cox et al., 2014; Verissimo et al., 2016). Indeed, patients bearing KRAS-mutant CRC result to be unresponsive to targeted-therapy, such as monoclonal antibody directed against EGFR (cetuximab and panitumumab), and still now, no effective therapeutic options are available. In fact, despite several improvements have been achieved, the survival rate at 5 years post diagnosis remains very poor (Bardelli and Siena, 2010; Brenner et al., 2014). Recently, Yun and colleagues reported that high-dose vitamin C is effective in killing KRAS mutant CRC cells, arising the possibility that vitamin C could have a potential therapeutic use for the treatment of this aggressive tumor type (Yun et al., 2015).

The anti-cancer properties of high-dose vitamin C have been associated with controversial results. In 1976, vitamin C was proposed by Cameron and Pauling as an anti-tumoral agent, however two randomized clinical trials failed to demonstrate any beneficial effect of oral-administered vitamin C on cancer patient survival (Cameron and Pauling, 1976; Creagan et al., 1979; Moertel et al., 1985). These contradictory outcomes are explained, at least in part, by the different administration route. In fact, growing evidence sustains that vitamin C requires to be delivered intravenously in order to bypass the gastric barrier and achieve plasma millimolar concentrations, which are toxic to cancer cells (Padayatty et al., 2010; Chen et al., 2008, Stephenson et al., 2013). For this reason, several studies are now focusing on vitamin C mechanism of action and on its effectiveness as anti-cancer drug, through several pre-clinical and clinical trials (Hoffer et al., 2008; Ma et al., 2014; Schoenfeld et al., 2017, NCT02344355; NCT03146962; NCT02420314; NCT01752491). The present invention identifies non-toxic but highly effective interventions with the potential to result in cancer free survival for the treatment of KRAS mutant cancers. Given the tolerability and efficacy of FMD and vitamin C as anti-cancer agents, inventors evaluated their combination as potential novel therapeutical option for colorectal cancer patients.

FMD/STS Sensitizes KRAS-Mutant CRC to Vitamin C Toxicity

In Summary Present Data Show that
STS is able to act synergistically with vitamin C in causing toxicity to a wide range of KRAS mutant cell lines derived from colorectal, lung and pancreatic cancers, leaving unaffected KRAS wild type prostate and ovary cancer cells, normal colon cells and normal fibroblasts. These findings suggest that KRAS oncogenic reprogramming is required for the FMD-mediated sensitization to vitamin C toxicity.
FMD cycles combined with pharmacological vitamin C treatment are more effective than vitamin C and FMD as single interventions in delaying tumor progression. Moreover, the tolerability of FMD and vitamin C combo therapy observed in both mouse strains, underlines its potential use in the clinic.

Vitamin C Toxicity in Cell Culture Systems

The in vitro cytotoxic action of vitamin C is object of debate. Several reports have shown that medium components contribute in determining vitamin C-dependent hydrogen peroxide generation (Clement et al., 2001; Mojic et al., 2014). Alpha-ketoglutarate and pyruvate, which directly interact with hydrogen peroxide, are able to quench its action (Nath et al., 1995). In addition, also different concentrations of iron, present in different commercially available growth media, could interfere with hydrogen peroxide production (Mono et al., 2014). Cell confluency is another important factor to be considered in evaluating vitamin C anti-cancer effect in vitro (Spitz et al., 1987). Cells are very often treated with high-dose vitamin C (>1 mM) at very low confluency, thus causing a massive and rapid cell death. Although in vivo vitamin C reaches millimolar concentrations, the in vitro cytotoxic effect could be overestimated.

In summary present data show that
Cell treatment at 40-50% confluency with vitamin C at a high dose (defined as >0.3 mM), but lower than 1 mM, best mimic the in vivo effect on tumor progression reduction.

In the present experimental system, CTR and STS media have identical composition, with the exception of glucose and serum concentration, which are responsible for the mimicking of the ad libitum feeding or fasting. Moreover, previous reports have shown that vitamin C toxicity is dependent on serum level, arising the possibility that this factor could be responsible for the enhancement of vitamin C toxicity mediated by STS condition. However, published data are controversial. In fact, it has been shown that serum levels can either enhance or inhibit vitamin C toxicity in different cell lines (Chen et al., 2005; Mojic et al., 2014).

Importantly, present data support that the observed phenotype is not a medium artefact. In fact, the intracellular iron chelation, mediated by DFO, is able to rescue cell death induction, indicating that the observed effect is intracellular and does not depend on extracellular media components. Moreover, modulation of HO-1 expression by pharmacological and genetic intervention, indicates that STS sensitizes cells to vitamin C through a genetic program.

Finally, the in vitro results were confirmed by in vivo findings, supporting the consistency of my in vitro condition as a good model system to study the effect of STS.

STS/FMD-Mediated Sensitization to Vitamin C is Dependent on ROS Generation

The molecular mechanism responsible for cancer cell-selective toxicity of vitamin C and the higher susceptibility of KRAS-mutant tumors, is still not completely elucidated.

Recently, Yun and colleagues reported that the overexpression of glucose transporter GLUT1 in KRAS-mutant tumors contributes to the increased uptake of the oxidized form of vitamin C (DHA) and thus its enhanced toxicity. In fact, it has been shown that under low glucose concentrations DHA uptake is increased and consequently cell death. However, GLUT1 overexpression is not sufficient to sensitize KRAS wild type cancer cells to vitamin C toxicity, suggesting that an oncogenic-reprogramming is required. Indeed, it has been found that intracellular DHA reduction to vitamin C depletes the glutathione reservoir causing an increase in ROS levels which in turn inhibits GAPDH. GAPDH inhibition results to be lethal in the highly glycolytic KRAS mutant cancer cells, leading to energetic crisis and cell death.

Although inventors have not investigated the activity status of GAPDH in my model system, they found that STS and vitamin C combo treatment induces markers of metabolic stress, in particular causing the increase in AMPK activity (measured by its phosphorylation level), which indicates a drop of ATP levels. This energetic crisis could be compatible with glycolysis inhibition, however the effect of STS and vitamin C on glycolytic pathways requires additional studies.

Consistent with previous findings, ROS accumulation has a central role in STS-mediated effects. Present data indicate that KRAS mutant CRC cells show an increase in ROS generation upon STS condition. However, despite the STS-induced effects, cancer cells do not show either DNA damage or death induction, possibly because of the up-regulation of a set of anti-oxidant enzymes, which can partially explain the limited efficacy of FMD in retarding tumor progression. The dramatic increase in ROS levels upon STS and vitamin C combo treatment, which induces both DNA damage and cell death, suggest that cancer cells are no longer able to counteract ROS generation. Indeed, the ability of anti-oxidants (GSH, NAC), as well as hydrogen peroxide and anion superoxide scavengers (catalase and MnTMPyP) to reverse cell death induction, support the hypothesis that oxidative stress is the causative factor in STS-mediated sensitization to vitamin C

Present data, together with previously published work on the role of anti-oxidants in reverting vitamin C toxicity to cancer cells, have important implication for the clinic (Chen et al., 2011; Yun et al., 2015). In fact, pro-oxidant therapies, such as chemotherapy or pharmacological vitamin C, are very often co-administered with anti-oxidant agents, which could interfere with the treatment (Chen et al., 2011).

STS/FMD-Mediated Sensitization to Vitamin C Requires HO-1 Down-Regulation

Despite controversial results, a growing body of studies has consistently shown that high-dose vitamin C exerts its cytotoxicity to cancer cells by a hydrogen peroxide-dependent mechanism (Chen et al., 2005; Chen et al., 2007; Chen et al., 2008; Du et al., 2010; Ma et al., 2014).

However, normal cells are protected since they may both generate less hydrogen peroxide and maintain high antioxidant defenses. On the contrary, cancer cells, which are characterized by low expression level of anti-oxidant enzymes and increased ROS, are sensitive to vitamin C associated toxicity (Liu et al., 2004; Oberley, 2005; Du et al., 2012; Doskey et al., 2016). In fact, ROS-disruption of iron metabolism increases the level of labile iron, which in turn reacts with hydrogen peroxide to generate hydroxyl radical, through the Fenton reaction chemistry, consequently causing oxidative damages and cell death (Schoenfeld et al., 2017). Accordingly, it has been shown that the increase in ROS and LIP levels obtained through deletion of the mitochondrial SOD gene, makes tumor cells more sensitive to vitamin C toxicity (Schoenfeld et al., 2017). Interestingly, evidence indicating that KRAS-driven cancers show higher level of ROS and LIP compared to KRAS wild type tumors, could partially explain their higher susceptibility to vitamin C toxicity (Kakhlon et al., 2002; Yang and Stockwell, 2008; Weinberg et al., 2010, Yun et al., 2015; Schoenfeld et al., 2017).

Here, inventors provide evidence showing that vitamin C treatment is able to up-regulate the expression level of the inducible-stress responsive protein heme-oxygenase-1 (HO-1). HO-1 catabolizes heme generating CO, biliverdin and free iron, which in turn induces ferritin expression. All these products show anti-oxidant and anti-inflammatory properties making HO-1 an anti-apoptotic and pro-survival enzyme (Was et al., 2010). It has been found that HO-1 is often overexpressed in tumors, in particular in response to chemotherapy, thus representing a mechanism of resistance and a poor prognostic factor for cancer patients (Was et al., 2010; Muliaditan et al., 2018).

Present in vitro and in vivo data show that, despite vitamin C is safe to normal tissues, its anti-cancer effect in reducing tumor progression is limited (Hoffer et al., 2008; Padayatty et al., 2010; Stephenson et al., 2013; Welsh et al., 2013; Ma et al., 2014). The induction of HO-1 upon vitamin C administration indicates that it may represent a possible mechanism of oxidative stress defence that cancer cells put in place in order to limit vitamin C effectiveness. In accordance with the described mechanism of vitamin C action, HO-1 up-regulation, by inducing ferritin expression, may limit free iron pool, and consequently Fenton chemistry and oxidative damages.

Present data show that FMD is able to revert vitamin C-mediated up-regulation of HO-1 both in vitro and in vivo. These findings raise the possibility that FMD potentiates vitamin C toxicity by impairing HO-1 pro-survival action. In support of this hypothesis, modulation of HO-1 expression dictates cancer cell susceptibility to vitamin C. In fact, HO-1 activation is able to revert STS-mediated cell death upon vitamin C co-treatment, whereas HO-1 pharmacological inhibition, as well as genetic silencing, is able to sensitize cancer cells to its toxicity in nutrient-rich condition.

As a consequence of the decrease in HO-1 level, FMD and vitamin C co-treatment causes ferritin down-regulation, which is accompanied by an increase in free ferrous iron. Supporting the role of high ferrous iron level in enhancing pro-oxidant reactions, intracellular iron chelation is able to revert STS-mediated cell death upon vitamin C treatment.

These results show the connection between the level of free iron in cancer cells and their susceptibility to vitamin C toxic action. Thus FMD, increasing ferrous iron, possibly through the reduction of HO-1 and ferritin level, exacerbates Fenton chemistry, leading to an enhanced toxicity.

FMD and Vitamin C Combo Treatment Potentiates Standard Chemotherapy Efficacy in KRAS Mutant CRC Mouse Models

Given the promising results on the safety and effectiveness of pharmacological vitamin C integration in standard therapy, inventors evaluated whether a more potent intervention as FMD and vitamin C combo treatment, could further potentiate chemotherapy efficacy.

The standard care for KRAS mutant CRC patients often includes oxaliplatin, a DNA synthesis inhibitor, with limited efficacy. Importantly, inventors found that FMD-like condition is able to sensitize KRAS-mutant CRC cells to oxaliplatin toxicity. Notably, FMD and vitamin C combinatorial treatment, further potentiates chemotherapy effectiveness in delaying tumor progression in two different mouse models.

Present results on three patients bearing KRAS mutant colorectal cancer, show the tolerability, feasibility and possibly efficacy of FMD cycles in combination with standard chemotherapy. Collectively, these data indicate that FMD and vitamin C co-treatment could represent a promising therapeutic option to enhance the efficacy of standard therapy, that still now show a very limited impact on survival of patients bearing KRAS-mutant cancer.

Collectively, present results show that FMD is a potent non-toxic intervention able to enhance the efficacy of pharmacological vitamin C in KRAS mutant CRC cells.

The proposed mechanism of action responsible for FMD-mediated sensitization to vitamin C involves the down-regulation of the inducible stress-responsive protein HO-1 (FIG. 31). FMD, by reverting vitamin C-dependent HO-1 induction, causes an increase of free ferrous ions. The boost of free iron levels, together with the FMD-mediated ROS increase, possibly enhance Fenton chemistry, finally leading to oxidative damage and cell death. The expansion of ferrous ions seems to occur through ferritin down-regulation, however it can't be excluded that the FMD-induced ROS could also be partially involved in iron metabolism disruption. Thus, future analyses are required to elucidate all these possibilities.

Then, FMD and vitamin C combination treatment represents a safe therapeutic option which can be easily integrated with standard therapy, to ameliorate the prognosis for patients bearing KRAS-driven cancers. Furthermore, present clinical results support the use of the combination of FMD, vitamin C and oxaliplatin triple treatment for KRAS mutant CRC patients.

### Example 3

Short-Term-Starvation Effect on KRAS Mutant CRC Sensitivity to High-Dose Vitamin C Toxicity

Stable Isotope Labelling with Amino Acids (SILAC) Analysis of Proteome Alteration of KRAS Mutant CRC Cells Upon Short Term-Starvation

Inventors performed a SILAC (stable isotope labeling with amino acids in cell culture) coupled to LC-MS/MS (Liquid chromatography-mass spectrometry/mass spectrometry) analysis on HCT116 cells, grown in CTR or STS condition.

STS condition, triggers considerable alterations in the protein expression profile of HCT116 KRAS mutant CRC cell line, compared to CTR. The inventors identified 308 (p<0.05) proteins that are statistically significantly (p<0.05) altered in their expression. In particular, 167 proteins were up-regulated (fold change >−1), whereas 141 were down-regulated (fold change <−1) upon STS.

Statistically significative proteins (red dots) were presented as scatter plot for graphical visualization (FIG. 32).

Next, lists of up-regulated and down-regulated proteins in STS condition were analysed by using Enrichr tool, a comprehensive gene set enrichment analysis web server (http://amp.pharm.mssm.edu/Enrichr) (Kuleshov et al., 2016).

Notably, enrichment analysis of gene ontology (GO) Molecular Function gene set library of up-regulated proteins upon 48 hours of STS, reveals a significative enrichment in molecular processes involved in anti-oxidant response (oxidoreductase activity, acting on CH—CH group of donors [GO:0016628], p=0.001496; oxidoreductase activity, acting on CH—OH group of donors [GO:0016616], p=0.005658; oxidoreductase activity, acting on the aldehyde or ketone (oxo) group of donors [GO:0016620], p=0.03018) (FIG. 33). The list of proteins involved in the anti-oxidant response [GO:0016209] is also reported (Table 1).

Enrichment analysis of GO Molecular Function gene set library of down-regulated proteins upon 48 hours of STS is listed in FIG. 34 (FIG. 34).

TABLE 1
Proteins with anti-oxidant activity up-regulated upon STS.
List of proteins with anti-oxidant properties [GO: 0016209] up-regulated
in KRAS mutant HCT116 grown for 48 hours in STS condition.

| Gene Symbol | Protein name |
|---|---|
| GSTO1 | Glutathione S-transferase omega-1 |
| PRDX2 | Peroxiredoxin-2 |
| APOM | Apolipoprotein M |
| MGST1 | Microsomal glutathione S-transferase 1 |
| PRDX5 | Peroxiredoxin-5, mitochondrial |
| NQO1 | NAD(P)H dehydrogenase [quinone] 1 |
| SOD1 | Superoxide dismutase [Cu—Zn] |

Holo-Transferrin, but not Apo-Transferrin, is Able to Reverse STS and Vitamin C Cytotoxic Effect

Iron bound-transferrin (holo-transferrin) and iron-free transferrin (apo-transferrin) are fundamental components present in the serum, which sustain cell growth in culture condition (Trowbridge and Shackelford, 1986)

For this reason, inventors tested whether transferrin supplementation could reverse cell death induction mediated by STS and vitamin C cotreatment. Interestingly, they found that only holo-transferrin, but not apo-transferrin, is able to rescue the observed cytotoxicity (FIG. 35).

Materials and Methods

Medium Dissection Experiments

For medium dissection experiments, cells were seeded in 12-well plate (34′000 cells per well) in CTR medium (1 g/l glucose, 10% FBS). The day after, cells were rinsed twice in 1×PBS, and grown in the following experimental conditions:
CTR medium: DMEM no glucose (Life Technologies, #11966025) supplemented with 1 g/l glucose (Sigma-Aldrich, #G8769) and 10% FBS (Biowest, Cat. #: S1810)
STS medium: DMEM no glucose (Life Technologies, #11966025) supplemented with 0.5 g/l glucose (Sigma-Aldrich, #G8769) and 1% FBS (Biowest, Cat. #: S1810)
Low glucose and normal serum medium: DMEM no glucose (Life Technologies, #11966025) supplemented with 0.5 g/l glucose (Sigma-Aldrich, #G8769) and 10% FBS (Biowest, Cat. #: S1810)
Normal glucose and low serum medium: DMEM no glucose (Life Technologies, #11966025) supplemented with 1 g/l glucose (Sigma-Aldrich, #G8769) and 1% FBS (biowest, Cat. #: S1810)
High glucose and low serum medium: DMEM no glucose (Life Technologies, Cat. #: 11966025) supplemented with 4.5 g/l glucose (Sigma-Aldrich, Cat. #: G8769) and 1% FBS (Biowest, Cat. #: S1810)

For serum dissection experiments, STS media was supplemented with IGF-1 (PeproTech, Cat. #: 100-11) (final concentration of 250 ng/ml), EGF (Biomol, Cat. #: BPS-90201-3) (final concentration of 200 ng/ml) and insulin (Sigma-Aldrich, Cat. #: 11376497001) (final concentration of 100 ng/ml). Essential amino acids (Life Technologies, Cat. #: 11130) were supplemented as 2× concentration compared to standard media, L-serine (Sigma Aldrich, Cat. #: S4311) was supplemented to a final concentration of 5 mM, L-glutamine (Biowest, Cat. #: X0550) was supplemented to a final concentration of 2 mM. Holo-transferrin (Sigma Aldrich, Cat. #: T0665) and apo-transferrin (Sigma Aldrich, Cat. #: T2252) were supplemented to a final concentration of 25 mg/ml.

After 24 hours, media were refreshed to ensure that glucose and serum levels were not completely exhausted, and after medium pH stabilization, cells were treated with 350 μM vitamin C or vehicle (deionized water) (sodium ascorbate, Sigma-Aldrich, Cat. #: A4034) for the next 24 hours. Cell death was measured as indicated.

Non-Essential Amino Acid and Glutamine Deprivation Experiments

For measurement of the contribution of non-essential amino acids and glutamine in sustaining HCT116 growth, cells were seeded in minimal essential medium (MEM, no phenol red, 1 g/L glucose) (Life Technologies, Cat. #: 51200), supplemented with 1% NEAA, 1% glutamine, 1% penicillin/streptomycin and 10% dialyzed serum (Life Technologies, Cat. #:26400044). The use of dialyzed serum is required, since it is designed to reduce the concentrations of small molecules, among which amino acids. In this way, it is possible to control the exact concentration of amino acids in the media, without variability due to the serum amino acidic content. Each single non-essential amino acid was purchased from Sigma-Aldrich. The day after, cells were rinsed twice in 1×PBS and grown in the followed experimental conditions for 48 hours:
MEM supplemented with 10% dialyzed FBS, all NEAA and glutamine
MEM supplemented with 10% dialyzed FBS, all NEAA, without glutamine
MEM supplemented with 10% dialyzed FBS, glutamine, all NEAA except L-serine
MEM supplemented with 10% dialyzed FBS, glutamine, all NEAA except L-proline
MEM supplemented with 10% dialyzed FBS, glutamine, all NEAA except L-glutamic acid
MEM supplemented with 10% dialyzed FBS, glutamine, all NEAA except L-aspartic acid
MEM supplemented with 10% dialyzed FBS, glutamine, all NEAA except L-asparagine
MEM supplemented with 10% dialyzed FBS, glutamine, all NEAA except L-alanine
MEM supplemented with 10% dialyzed FBS, glutamine, all NEAA except L-glycine
MEM supplemented with 10% dialyzed FBS, glutamine, without all NEAA

At the end of the experiment, cell viability was evaluated by MTT assay.

SILAC and Analysis of Proteins by LC-MS-MS

HCT116 cells were grown in medium for SILAC (DMEM without lysine and arginine, Life Technologies, Cat. #: A2493901) supplemented with 10% dialyzed serum, 1% penicillin/streptomycin, 2 mM glutamine, 1% non-essential amino acids and isotopically labelled forms of arginine and lysine (heavy) or normal arginine and lysine (light). Cells were grown in “heavy” or “light” media for 6 passages to allow amino acids incorporation. Heavy lysine and arginine incorporation was checked by LS-MS-MS and results to be more than 95%.

Cells that have incorporated heavy amino acids arginine and lysine were then grown in SILAC DMEM media supplemented with 1 g/l glucose and 10% dialysed serum (CTR), whereas cells that have incorporated “light” amino acids, were then grown in SILAC DMEM media supplemented with 0.5 g/l glucose and 1% dialysed serum (STS), SILAC heavy and light labelled cells were lysed with in Urea buffer (8 M Urea, 10 mM Tris-HCl, 150 mM NaCl, 1 mM EDTA), and proteins quantified by Bradford. CTR (heavy) and STS (light) samples were mixed proportionally to 1:1 based on protein extract amount. About 30 μg of mixed proteins for each sample were reduced by TCEP, alkylated by chloroacetamide, digested by Lys-C and Trypsin (Kulak et al., 2014) and peptides desalted on StageTip C18 (Rappsilber et al., 2003). 1 μl of each sample was analyzed as technical replicate on a LC (liquid-chromatography)-ESI-MS-MS quadrupole Orbitrap QExactive-HF mass spectrometer (Thermo Fisher Scientific). Peptides separation was achieved on a linear gradient from 93% solvent A (2% ACN, 0.1% formic acid) to 60% solvent B (80% acetonitrile, 0.1% formic acid) over 110 minutes and from 60 to 100% solvent B in 8 min at a constant flow rate of 0.25 μl/min on UHPLC Easy-nLC 1000 (Thermo Scientific) connected to a 23-cm fused-silica emitter of 75 μm inner diameter (New Objective, Inc. Woburn, Mass., USA), packed in-house with ReproSil-Pur C18-AQ 1.9 μm beads (Dr Maisch Gmbh, Ammerbuch, Germany) using a high-pressure bomb loader (Proxeon, Odense, Denmark). MS data were acquired using a data-dependent top 20 method for HCD fragmentation. Survey full scan MS spectra (300-1650 Th) were acquired in the Orbitrap with 60000 resolutions, AGC target 3^{e6}, IT 20 ms. For HCD spectra, resolution was set to 15000 at m/z 200, AGC target 1^{e5}, IT 80 ms; NCE 28% and isolation width 2.0 m/z. For quantitative proteomic, raw data were processed with MaxQuant (ver. 1.5.2.8) searching against the database uniprot_cp_human_2015_03 setting labelling Arg10 and Lys8, trypsin specificity and up to two missed cleavages. Cysteine carbamidomethyl was used as fixed modification, methionine oxidation and protein N-terminal acetylation as variable modifications. Mass deviation for MS-MS peaks was set at 20 ppm. The peptides and protein false discovery rates (FDR) were set to 0.01; the minimal length required for a peptide was six amino acids; a minimum of two peptides and at least one unique peptide were required for high-confidence protein identification. The lists of identified proteins were filtered to eliminate reverse hits and known contaminants.

Statistical analyses were done using the Perseus program (Version 1.5.1.6) in the MaxQuant environment. Ratios H/L and proteins intensity normalized were transformed in log 2 and log 10 respectively. Significance B outlier test was applied and statistical significance based on magnitude fold-change was established at p-value <0.05.

### Example 4

FMD Cycles Delay Tumor Progression and Enhance Vitamin C Anti-Cancer Effect in KRAS Mutant CRC Mouse Models

To further confirm the efficacy of the proposed therapeutic intervention in in vivo settings, inventors have taken advantage from an orthotopic mouse model. CT26-KRAS-mutant—expressing luciferase were injected submucosally into the distal, posterior rectum, as previously described (Donigan et al., 2010). The metastatic colon cancer model using a non-operative transanal rectal injection allows the evaluation of tumor progression in its own environment and currently represents the most accurate orthotopic model with several advantages compared to the standard caecum injection (Donigan et al., 2010). Present data shows that FMD and vitamin C combo treatment represents the most effective intervention in reducing cancer progression, in agreement with the other in vivo models presented herein (FIG. 36).

Alterations in Intracellular Iron Levels Induced by FMD are Responsible for the Enhancement of Vitamin C Efficacy

To characterize how FMD/STS affect iron metabolism through HO-1/FTH pathways and modulate cancer cell sensitivity to vitamin C, inventors analysed the effect of serum transferrin in mediating STS effects. Addition of holo-transferrin (iron-bound form) but not apo-transferrin (iron-free form) reversed STS+vitamin C-mediated toxicity (FIG. 37).

These results are consistent with the concept that iron levels in the serum are important in mediating the STS effect. In fact, in agreement with present in vitro findings, in vivo FMD+Vitamin C reduced blood levels of transferrin bound iron (FIG. 50h). Present data further support the role of iron as a key factor in the serum whose reduction was responsible for the synergism of FMD and vitamin C

In Vivo Procedures

Mouse Models

For the orthotopic model, the animal protocol was approved by the Institutional Animal Care and Use Committee (IACUC) of the University of Southern California. Female BALB/c mice (8 weeks old, strain 000651-Jackson Laboratory) were anesthetized with isoflurane anaesthesia. Mice then received a gentle anal dilation using blunt-tipped forceps at the anal opening. A 29-gauge syringe was used to inject 2.5×10⁴ CT26-luc cells (SC061-LG GenTarget Inc), suspended in saline, submucosally into the distal, posterior rectum. Seven days later, mice were randomly divided in the different experimental groups. Twenty-one days post injections mouse imaging was performed using the Xenogen IVIS-200 System. Mice were anesthetized by isoflurane anaesthesia and luciferin (50 mg/kg body weight) was administered via intra-peritoneal injections and animals were subjected to Bioluminescence Imaging (BLI) at the USC Small Animal Imaging Center.

At the end of the experiments, mice were euthanized by using CO₂.

Statistical Analysis

For orthotopic mouse model experiment outliers were removed according to ROUT method (ROUT=1%). A confidence interval of 95% (P values≤0.05) were considered significant. For survival analysis, the Log-rank (Mantel-Cox) test was performed.

Viability Assay

For cell death measurement, cells were seeded in 12-well plates so that at the moment of vitamin C treatment, cells reach 40% of confluence. 24 hours after seeding, cells were rinsed twice in PBS and then grown in STS medium or STS medium supplemented with apo-transferrin (Sigma Aldrich, Cat. #: T2252, 0.3 mg/ml) or holo-transferrin (Sigma Aldrich, Cat. #: T0665, 0.3 mg/ml)

After 24 hours media were refreshed and cells were treated with vitamin C (350 μM).

At the end of the experiment cells were harvested by trypsinization, centrifuged and resuspended for a final concentration of 1×10⁶ cells per ml. Cell viability was measured by Muse viability assay kit (Merck Millipore, Cat. #: MCH100102).

In Vivo Iron-Bound Transferrin Measurement

For iron-bound transferrin measurement, mouse blood was collected from the heart of mice sacrificed at the end of 2^{nd} FMD cycle and 24 hours post-refeeding. Blood was incubated at room temperature (25° C.) for at least 30 minutes to clot and then centrifuged for 15 minutes at 2,000×g (4° C.). Collected serum was aliquoted and stored at −80° C. Iron-bound transferrin was measured by Serum Iron Assay kit (Biovision, #K392) according to manufacturer protocol.

### REFERENCES

Aguilera O, et al., Oncotarget. 2016 Jul. 26; 7(30):47954-47965.
Bardelli A, Siena S. J Clin Oncol. 2010 Mar. 1; 28(7):1254-61.
Berberat P O, et al., Clin Cancer Res. 2005 May 15; 11(10):3790-8.
Brandhorst S, et al., Cell Metab. 2015 Jul. 7; 22(1):86-99.
Brenner H, Kloor M, Pox C P. Lancet. 2014 Apr. 26; 383(9927):1490-1502.
Busserolles J, et al., Int J Biochem Cell Biol. 2006; 38(9):1510-7.
Cameron E, Pauling L. Proc Natl Acad Sci USA. 1976 October; 73(10):3685-9.
Cancer Chemother Pharmacol. 2013 March; 71(3):765-75.
Cardaci, S., et al., J. Cell Sci. 125, 2115-2125.
Chelikani, P., et al., Cell. Mol. Life Sci. 2004, 61, 192-208.
Chen Q, et al., Proc Natl Acad Sci USA. 2007 May 22; 104(21):8749-54.
Chen Q, et al., Proc Natl Acad Sci USA. 2008 Aug. 12; 105(32):11105-9.
Clément, M. V., et al., Antioxid. Redox Signal. 3, 157-163.
Cox A D, et al., Nat Rev Drug Discov. 2014 November; 13(11):828-51.
Creagan E T, et al., N Engl J Med. 1979 Sep. 27; 301(13):687-90.
Di Biase S, et al., Cancer Cell. 2016 Jul. 11; 30(1):136-146.
Donigan M., et al., Surg Endosc. 2010 March; 24(3):642-7.
Doskey, C. M., et al., Redox Biol. 2016, 10, 274-284.
Du J, et al., Biochim Biophys Acta. 2012 December; 1826(2):443-57.
Du, J., et al., Clin Cancer Res. 16, 509-520.
Ferris, C. D., et al. Nature Cell Biology 1999, 1, 152-157.
Gonzales, S., et al., Dev Neurosci 2002, 24, 161-168.
Hardie, D. G., et al., Nat. Rev. Mol. Cell Biol. 2012, 13, 251-262.
Hoffer L J, et al., Ann Oncol. 2008 November; 19(11):1969-74.
Kakhlon, O., et al., Biochem. J. 2002 May; 363 (Pt 3):431-6.
Kim H R, et al., Lung Cancer. 2008 April; 60(1):47-56.
Kimball, S. R., 1999. Int. J. Cell Biol. 31, 25-29.
Kimmelman, A. C., 2015. Clinical Cancer Research 21, 1828-1834.
Lee C, Longo V D. Oncogene. 2011 Jul. 28; 30(30):3305-16.
Lee C, et al., Sci Transl Med. 2012 Mar. 7; 4(124):124ra27.
Lee C, et al., Cancer Res. 2010 Feb. 15; 70(4):1564-72.
Lievre A, et al., Cancer Res. 2006 Apr. 15; 66(8):3992-5.
Liu Z M, et al., Oncogene. 2004 Jan. 15; 23(2):503-13.
Longo V D, Mattson M P. Cell Metab. 2014 Feb. 4; 19(2):181-92.
Ma Y, et al., Sci Transl Med. 2014 Feb. 5; 6(222):222ra18.
Manning, B. D., Cantley, L. C., 2007. Cell 129, 1261-1274.
Moertel C G, et al., N Engl J Med. 1985 Jan. 17; 312(3):137-41.
Mojie, M., et al., Sci. Rep. 2014, 4, 5955.
Monti D A, et al., PLoS One. 2012; 7(1):e29794.
Muliaditan, T., et al., Clinical Cancer Research 2018, 24, 1617-1628.
Nath, K. A., et al., Am. J. Physiol. 1995, 268, C227-36.
Oberley, L. W., 2005. Biomed. Pharmacother. 59, 143-148
Padayatty S J, et al., Ann Intern Med. 2004 Apr. 6; 140(7):533-7.
Raffaghello L, et al., Proc Natl Acad Sci USA. 2008 Jun. 17; 105(24):8215-20.
Reczek, C. R., et al., Annu. Rev. Cancer Biol. 2017 1, 79-98
Safdie F M, et al., Aging (Albany N.Y.). 2009 Dec. 31; 1(12):988-1007.
Schoenfeld J D, et al., Cancer Cell. 2017 Aug. 14; 32(2):268.
Spitz, D. R., et al., J. Cell. Physiol. 1987, 131, 364-373.
Stephen A G, et al., Cancer Cell. 2014 Mar. 17; 25(3):272-81.
Stephenson C M, et al., Cancer Chemother Pharmacol. 2013 July; 72(1):139-46.
Torti, S. V., Torti, F. M. Nat. Rev. Cancer 2013 May; 13 (5): 342-55.
Tzatsos, A., et al., J Biol Chem. 2007, 282, 18069-18082.
Van Emburgh, et al., Nat Comms 2016, 7, 13665.
Verissimo C S, et al., Elife. 2016 Nov. 15; 5.
Was H, et al., Curr Drug Targets. 2010 December; 11(12):1551-70.
Wei M, et al., Sci Transl Med. 2017 Feb. 15; 9(377).
Weinberg, F., et al. Proc. Natl. Acad. Sci. U.S.A. 2010, 107, 8788-8793.
Welsh J L, et al., Cancer Chemother Pharmacol. 2013 March; 71(3):765-75.
Yang, W. S., et al., Chem. Biol. 2008 March; 15(3):234-45.
Yun J, et al., Science. 2015 Dec. 11; 350(6266):1391-6.

1. A method for the treatment of cancer, comprising administering a fasting mimicking diet and vitamin C to a patient in need thereof.

2. The method of claim 1, wherein said fasting mimicking diet is administered for a period of 24 to 190 hours.

3. The method of claim 1, wherein said fasting mimicking diet is administered for a period of 24 to 120 hours.

4. The method of claim 1, wherein said fasting mimicking diet is administered for approximately 5 days.

5. The method of claim 1, wherein said fasting mimicking diet is a diet wherein regular caloric intake reduced by 10% to 100%.

6. The method of claim 1, wherein said fasting mimicking diet is a diet wherein regular caloric intake reduced by 45% to 95%.

7. The method of claim 1, wherein said fasting mimicking diet is a diet wherein regular caloric intake reduced by approximately 50% to 70%.

8. The method of claim 1, wherein said fasting mimicking diet comprises a first period of 0 to 24 hours wherein caloric intake is a regular caloric intake reduced by 40-60%, followed by a second period of 24 to 144 hours wherein caloric intake is a regular caloric intake reduced by 60-95%.

9. The method of claim 8, wherein said first period lasts approximately 24 hours and said second period lasts approximately from 48 to 96 hours.

10. The method of claim 1, wherein said fasting mimicking diet comprises a reduced protein intake and/or a reduced simple carbohydrate intake and/or an increased complex carbohydrate intake and/or an increased unsaturated fat intake.

11. The method of claim 10, wherein said reduced protein intake is from 5 to 15% of total caloric intake, said increased complex carbohydrate intake is from 40 to 50% of total caloric intake, and said increased unsaturated fat intake is from 40 to 50% of total caloric intake.

12. The method of claim 11, wherein said reduced protein intake is approximately from 9 to 11% of total caloric intake, said increased complex carbohydrate intake is approximately from 43 to 47% of total caloric intake, said increased unsaturated fat intake is approximately from 44 to 46% of total caloric intake.

13. The method of claim 1, wherein said vitamin C is administered parenterally.

14. The method of claim 13, wherein said vitamin C is administered intravenously.

15. The method of claim 1, wherein said vitamin C is administered in an amount of approximately from 50 to 100 g. per dose.

16. The method of claim 1, wherein said vitamin C is administered three times per week.

17. The method of claim 1, wherein said fasting mimicking diet and vitamin C are combined with a further therapeutic intervention.

18. The method of claim 1, wherein said further therapeutic intervention is selected from the group consisting of: surgery, radiotherapy and a further therapeutic agent.

19. The method of claim 1, wherein said further therapeutic agent is a chemotherapeutic agent, optionally selected from the group consisting of: a DNA synthesis inhibitor, a monoclonal antibody and a Heme Oxygenase-1 (HO-1) inhibitor.

20. The method of claim 19, wherein said monoclonal antibody is a monoclonal antibody directed against EGFR.

21. The method of claim 19, wherein said chemotherapeutic agent is selected from the group consisting of: oxaliplatin, zinc protoporphyrin, 5-fluorouracil (5-FU), folinic acid, irinotecan, capecitabine, cetuximab, panitumumab, bevacizumab, FOLFOX, FOLFOXIRI, XELIRI, and XELOX.

22. The method of claim 1, wherein said cancer is a solid cancer, a RAS mutant cancer or a KRAS mutant cancer.

23. The method of claim 1, wherein said cancer is resistant to conventional therapy.

24. The method of claim 1, wherein said cancer is selected from the group consisting of: colorectal cancer, lung cancer, pancreatic cancer, colon cancer, rectal cancer, mucinous adenocarcinoma.

25. The method of claim 1, wherein said cancer is a KRAS mutant solid cancer.
